SEQUENCE LISTING (39 sequences)

SEQ ID NO: 1 - RNA, 96 nt, Artificial Sequence
  Synthetic Polynucleotide
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 2 - RNA, 10 nt, Artificial Sequence
  Synthetic Polynucleotide
aauacauccc

SEQ ID NO: 3 - RNA, 16 nt, Artificial Sequence
  Synthetic Polynucleotide
agcgcaagaagaaauc

SEQ ID NO: 4 - RNA, 14 nt, Artificial Sequence
  Synthetic Polynucleotide
cuuuggaguaugac

SEQ ID NO: 5 - RNA, 14 nt, Artificial Sequence
  Synthetic Polynucleotide
gucauacuccaaag

SEQ ID NO: 6 - RNA, 16 nt, Artificial Sequence
  Synthetic Polynucleotide
gauuucuucuugcgcu

SEQ ID NO: 7 - DNA, 14 bp, Artificial Sequence
  Synthetic Polynucleotide
gtcatactccaaag

SEQ ID NO: 8 - DNA, 16 bp, Artificial Sequence
  Synthetic Polynucleotide
gatttcttcttgcgct

SEQ ID NO: 9 - DNA, 20 bp, Artificial Sequence
  Synthetic Polynucleotide
ggttcacagtcggtcacatt

SEQ ID NO: 10 - RNA, 12 nt, Artificial Sequence
  Synthetic Polynucleotide
gauuucuucuug

SEQ ID NO: 11 - RNA, 10 nt, Artificial Sequence
  Synthetic Polynucleotide
gggauguauu

SEQ ID NO: 12 - RNA, 24 nt, Artificial Sequence
  Synthetic Polynucleotide
auaacuaagaacgacgaugacaca

SEQ ID NO: 13 - RNA, 24 nt, Artificial Sequence
  Synthetic Polynucleotide
acagucaucgucguucuuaguuau

SEQ ID NO: 14 - RNA, 16 nt, Artificial Sequence
  Synthetic Polynucleotide
ugaccgacugugaacc

SEQ ID NO: 15 - RNA, 16 nt, Artificial Sequence
  Synthetic Polynucleotide
gguucacagucgguca

SEQ ID NO: 16 - RNA, 55 nt, Artificial Sequence
  Synthetic Polynucleotide
guuuuagagcuaugcugaaaagcauagcaaguuaaaauaaggcuaguccguuauc

SEQ ID NO: 17 - RNA, 10 nt, Artificial Sequence
  Synthetic Polynucleotide
gccuacucau

SEQ ID NO: 18 - RNA, 16 nt, Artificial Sequence
  Synthetic Polynucleotide
gaguuguaauugugcc

SEQ ID NO: 19 - RNA, 14 nt, Artificial Sequence
  Synthetic Polynucleotide
uuguauaugugucc

SEQ ID NO: 20 - RNA, 13 nt, Artificial Sequence
  Synthetic Polynucleotide
ggacacauauaca

SEQ ID NO: 21 - RNA, 16 nt, Artificial Sequence
  Synthetic Polynucleotide
ggcacaauuacaacuc

SEQ ID NO: 22 - RNA, 16 nt, Artificial Sequence
  Synthetic Polynucleotide
ggcaccuaacauacac

SEQ ID NO: 23 - RNA, 16 nt, Artificial Sequence
  Synthetic Polynucleotide
guguauguuaggugcc

SEQ ID NO: 24 - RNA, 14 nt, Artificial Sequence
  Synthetic Polynucleotide
gguuaguaauguua

SEQ ID NO: 25 - RNA, 14 nt, Artificial Sequence
  Synthetic Polynucleotide
uaacauuacuaacc

SEQ ID NO: 26 - DNA, 38 bp, Artificial Sequence
  Synthetic Polynucleotide
ttccttaatggaggttgactccaatactaaggtaggat

SEQ ID NO: 27 - DNA, 38 bp, Artificial Sequence
  Synthetic Polynucleotide
atcctaccttagtattggagtcaacctccattaaggaa

SEQ ID NO: 28 - RNA, 32 nt, Artificial Sequence
  Synthetic Polynucleotide
ggagguugacuccaauacuaguuuuagagcua

SEQ ID NO: 29 - RNA, 65 nt, Artificial Sequence
  Synthetic Polynucleotide
uagcaaguuaaaauaaggcuaguccguuaucaacuugaaaaaaguggcaccgagucggugcuuuu

SEQ ID NO: 30 - RNA, 101 nt, Artificial Sequence
  Synthetic Polynucleotide
ggagguugacuccaauacuaguuuuagagcuagaaauagcaaguuaaaauaaggcuaguccguuaucaacuugaaaaaaguggcaccgagucggugcuuuu

SEQ ID NO: 31 - DNA, 10 bp, Artificial Sequence
  Synthetic Polynucleotide
cagtccttta

SEQ ID NO: 32 - DNA, 34 bp, Artificial Sequence
  Synthetic Polynucleotide
ggttgccaagcgcacctaatttcctaaaggactg

SEQ ID NO: 33 - RNA, 43 nt, Artificial Sequence
  Synthetic Polynucleotide
aauuucuacucuuguagauggaaauuaggugcgcuuggcaacc

SEQ ID NO: 34 - RNA, 53 nt, Artificial Sequence
  Synthetic Polynucleotide
gaaauauggauuacuugguagaacagcaaucuacucgaccugcaggcaugcaa

SEQ ID NO: 35 - RNA, 56 nt, Artificial Sequence
  Synthetic Polynucleotide
ccaccccaauaucgaaggggacuaaaacuagauugcuguucuaccaaguaauccau

SEQ ID NO: 36 - RNA, 12 nt, Artificial Sequence
  Synthetic Polynucleotide
gucauacuccaa

SEQ ID NO: 37 - RNA, 76 nt, Artificial Sequence
  Synthetic Polynucleotide
guuuuagagcuagaaauagcaaguuaaaauaaggcuaguccguuaucaacuugaaaaaguggcaccgagucggugc

SEQ ID NO: 38 - DNA, 500 bp, Artificial Sequence
  Synthetic Polynucleotide
tcgaggtcgcaggtatctttagggtagacatcttacggggagagcactaacccatcaacctgtacggttcacagtcggtcacattggggtactccttggcttttccgcttggcccggtctgttaggcccccgtagcgtgagtttcggccctgtgctgcccagtacgagctgattgaacatgggttgggggcctcgaaccgtcgaggaccccatagtacctcggagaccaagtgatttcttcttgcgcttttttgggaactatttcggggggcgagccctcatcgtctcttcgcggatgactcaacacgctagggacgtgaccgcgggcagtcgattccttcgatggttataaatcgcgtgcgctctatttgacgatcccttggcgcagaggtgctggccacgtgctaaattaaagcggctgcactactgtaaggtccgtcacggatgtgattccactctattggacggcggccaaagactaaagaccatctcgctcgaga

SEQ ID NO: 39 - DNA, 20 bp, Artificial Sequence
  Synthetic Polynucleotide
gatttcttcttgcgcttttt

(19) United States
(12) Patent Application Publication
(10) Pub. No.: US 2019/0233806 A1
(43) Pub. Date: Aug. 1, 2019

(54) CONDITIONAL ACTIVATION OF NUCLEIC ACID-GUIDED ENDONUCLEASES

(71) Applicant: President and Fellows of Harvard College, Cambridge, MA (US)

(72) Inventors: Nicolas Garreau de Loubresse, Cambridge, MA (US); Jongmin Kim, Cambridge, MA (US); Peng Yin, Brookline, MA (US)

(73) Assignee: President and Fellows of Harvard College, Cambridge, MA (US)

(21) Appl. No.: 16/312,199

(22) PCT Filed: Jun. 23, 2017
(86) PCT No.: PCT/US2017/038998

§ 371 Date: Dec. 20, 2018

Related U.S. Application Data
(60) Provisional application No. 62/429,120, filed on Dec. 2, 2016.
(60) Provisional application No. 62/354,016, filed on Jun. 23, 2016.

(51) Int. Cl. C12N 9/22; C12N 15/11; C12N 15/113

(52) U.S. Cl. CPC C12N 9/22; C12N 15/11; C12N 2310/3519; C12N 2310/20; C12N 15/113

(57) ABSTRACT

The present disclosure provides, in some embodiments, methods and compositions that use secondary nucleic acid structures for regulating RNA-guided endonuclease activity and/or DNA-guided endonuclease activity.

### RELATED APPLICATIONS

This application claims the benefit under 35 U.S.C. § 119(e) of U.S. provisional application No. 62/354,016, filed Jun. 23, 2016, and U.S. provisional application No. 62/429,120, filed Dec. 2, 2016, each of which is incorporated by reference herein in its entirety.

### FEDERALLY SPONSORED RESEARCH

This invention was made with government support under CCF-1317291 awarded by National Science Foundation. The government has certain rights in the invention.

### BACKGROUND

RNA-guided endonucleases such as CRISPR/Cas9, Cpf1, C2c2 use RNA molecules as guides to recognize and cleave complementary nucleic acid sequences (DNA or RNA). These “guide RNAs,” or “gRNAs,” provide the specificity of RNA-guided endonucleases. DNA-guided endonucleases, such as NgAgo, use DNA molecules as guides to recognize and cleave complementary nucleic acid sequences (e.g., DNA or RNA). These “guide DNAs,” or “gDNAs,” provide the specificity of DNA-guided endonucleases. RNA-guided and DNA-guided enzymes have been mainly used and engineered for genome editing, gene expression regulation, labeling and/or cleaving of specific nucleic acids.

### SUMMARY

Provided herein, in some embodiments, are engineered gRNAs (and associated methods) that are inactive (cannot bind to a cognate nuclease) and become active (can bind to a cognate nuclease) only when bound by a specific trigger nucleic acid sequence. Controlled activation of gRNAs permits control of cognate nuclease (e.g., Cas9, Cpf1 or C2c2) activity. Unexpectedly, results provided herein show that engineered gRNAs can be kept in an inactive state by introducing a duplex (double-stranded region) adjacent to a guide domain of the gRNA (the domain containing sequence complementary to a target sequence) (see, e.g., FIG. 9A).

Also provided herein, in some embodiments, are engineered gDNAs that can be activated by specific RNA sequences, which permits control of the activity of a cognate nuclease (e.g., NgAgo).

It should be understood that engineered gRNAs and gDNAs of the present disclosure may be expressed in vivo (e.g., in a subject) or produced in vitro and subsequently introduced into target cells (e.g., in a subject).

Thus, in one aspect, provided herein is a composition, comprising (a) an inactive guide ribonucleic acid (RNA) comprising a 5′ end, a 3′ end, and (i) an unpaired hairpin loop domain, (ii) a paired stem domain located adjacent to the unpaired hairpin loop domain and comprising a first subdomain complementary to and bound to a second subdomain, (iii) an unpaired toehold domain contiguous with the first subdomain of the paired stem domain, and (iv) an unpaired guide domain contiguous with the second subdomain of the paired stem domain that is capable of associating with an RNA-guided endonuclease when the first subdomain and second subdomain of (a)(ii) are not bound to each other; and optionally (b) a trigger nucleic acid comprising (i) an unpaired subdomain complementary to the first subdomain of the paired stem domain of the inactive gRNA and (ii) an unpaired subdomain complementary to the toehold domain of the inactive gRNA.

In some embodiments, the composition comprises both the inactive guide RNA and the trigger nucleic acid. In some embodiments, the composition further comprises the RNA-guided endonuclease. In some embodiments, the composition further comprises a target nucleic acid. In some embodiments, the guide domain of (a)(iv) above comprises a nucleotide sequence that is complementary to the target nucleic acid. In some embodiments, the RNA-guided endonuclease is Cas9, Cpf1 or C2c2.

In some embodiments, the unpaired toehold domain is located at the 5′ end of the inactive guide RNA. In some embodiments, the 3′ end of the inactive guide RNA comprises a scaffold formed by intramolecular nucleotide base pairing. In some embodiments, the unpaired subdomain (i) of the trigger nucleic acid is upstream from the unpaired subdomain (ii) of the trigger nucleic acid. In some embodiments, the unpaired toehold domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (ii) of the trigger nucleic acid is upstream from the unpaired subdomain (i) of the trigger nucleic acid.

In another aspect, the disclosure provides a method comprising incubating in reaction buffer in the presence of a target nucleic acid and an RNA-guided nuclease, any of the inactive guide RNAs described above and any of the trigger nucleic acids described above to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced first and the trigger may be introduced second, or vice versa.

In another aspect, the disclosure provides a composition, comprising: (a) an inactive guide ribonucleic acid (RNA) comprising a 5′ end, a 3′ end, and (i) an unpaired hairpin loop domain, (ii) a paired stem domain located adjacent to the unpaired hairpin loop domain and comprising a first subdomain complementary to and bound to a second subdomain, and (iii) an unpaired guide domain contiguous with the second subdomain of the paired stem domain that is capable of associating with an RNA-guided endonuclease when the first subdomain and second subdomain of (a)(ii) are not bound to each other; and optionally (b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA.

In some embodiments, the composition comprises both the inactive guide RNA and the trigger nucleic acid. In some embodiments, the composition further comprises the RNA-guided endonuclease. In some embodiments, the composition further comprises a target nucleic acid. In some embodiments, the guide domain of (a)(iii) comprises a nucleotide sequence that is complementary to the target nucleic acid. In some embodiments, the RNA-guided endonuclease is Cas9, Cpf1 or C2c2.

In some embodiments, the first domain is located at the 5′ end of the inactive guide RNA. In some embodiments, the 3′ end of the inactive guide RNA comprises a scaffold formed by intramolecular nucleotide base pairing. In some embodiments, the unpaired subdomain (i) of the trigger nucleic acid is upstream from the unpaired subdomain (ii) of the trigger nucleic acid. In some embodiments, the first domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (ii) of the trigger nucleic acid is upstream from the unpaired subdomain (i) of the trigger nucleic acid.

Provided herein, in one aspect, is a method comprising incubating in reaction buffer in the presence of a target nucleic acid and an RNA-guided nuclease, any of the inactive guide RNAs described above and any of the trigger nucleic acids described above to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced before or after the trigger nucleic acid.

In another aspect, the disclosure provides a composition, comprising: (a) an inactive guide ribonucleic acid (RNA) comprising a 5′ end, a 3′ end, and (i) an unpaired hairpin loop domain, (ii) a paired stem domain located adjacent to the unpaired hairpin loop domain and comprising a first subdomain complementary to and bound to a second guide subdomain that, when not bound to the first subdomain, associates with an RNA-guided endonuclease, and (iii) an unpaired toehold domain contiguous with the first subdomain of the paired stem domain; and optionally (b) a trigger nucleic acid comprising (i) an unpaired subdomain complementary to the first subdomain of the paired stem domain of the inactive gRNA and (ii) an unpaired subdomain complementary to the toehold domain of the inactive gRNA. In some embodiments, the composition comprises both the inactive guide RNA and the trigger nucleic acid. In some embodiments, the composition further comprises the RNA-guided endonuclease. In some embodiments, the composition further comprises a target nucleic acid. In some embodiments, the second subdomain of (a)(ii) above comprises a nucleotide sequence that is complementary to a target gene of interest. In some embodiments, the RNA-guided endonuclease is Cas9, Cpf1 or C2c2.

In some embodiments, the unpaired toehold domain is located at the 5′ end of the inactive guide RNA. In some embodiments, the 3′ end of the inactive guide RNA comprises a scaffold formed by intramolecular nucleotide base pairing. In some embodiments, the unpaired subdomain (i) of the trigger nucleic acid is upstream from the unpaired subdomain (ii) of the trigger nucleic acid described above. In some embodiments, the unpaired toehold domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (ii) of the trigger nucleic acid is upstream from the unpaired subdomain (i) of the trigger nucleic acid.

In another aspect, the disclosure provides a method comprising incubating in reaction buffer in the presence of a target nucleic acid and an RNA-guided nuclease, any of the inactive guide RNAs described above and any of the trigger nucleic acids described above to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced before or after the trigger nucleic acid.

Provided herein, in one aspect is a composition, comprising: (a) an inactive guide ribonucleic acid (RNA) comprising a 5′ end, a 3′ end, and (i) an unpaired hairpin loop domain, (ii) a paired stem domain located adjacent to the unpaired hairpin loop domain and comprising a first subdomain complementary to and bound to a second guide subdomain that, when not bound to the first subdomain, associates with an RNA-guided endonuclease, and (iii) an unpaired toehold domain contiguous with the first subdomain of the paired stem domain; and optionally (b) a first trigger nucleic acid comprising (i) an unpaired subdomain complementary to the first subdomain of the paired stem domain of the inactive gRNA and (ii) an unpaired subdomain; and further optionally (c) a second trigger nucleic acid comprising (i) an unpaired subdomain complementary to the unpaired subdomain of (b)(ii) and (ii) an unpaired subdomain complementary to the toehold domain of the inactive gRNA.

In some embodiments, the composition comprises the inactive guide RNA, the first trigger nucleic acid, and the second trigger nucleic acid. In some embodiments, the composition comprises the inactive guide RNA and the first trigger nucleic acid. In some embodiments, the composition comprises the inactive guide RNA and the second trigger nucleic acid. In some embodiments, the composition comprises the first trigger nucleic acid and the second trigger nucleic acid. In some embodiments, the composition further comprises the RNA-guided endonuclease. In some embodiments, the composition further comprises a target nucleic acid. In some embodiments, the second subdomain of (a)(ii) above comprises a nucleotide sequence that is complementary to the target nucleic acid. In some embodiments, the RNA-guided endonuclease is Cas9, Cpf1 or C2c2.

Provided herein, in one aspect, is a composition, comprising: (a) an inactive guide ribonucleic acid (RNA) comprising a 5′ end, a 3′ end, and (i) an unpaired hairpin loop domain, and (ii) a paired stem domain located adjacent to the unpaired hairpin loop domain and comprising a first subdomain complementary to and bound to a second guide subdomain that, when not bound to the first subdomain, associates with an RNA-guided endonuclease; and optionally (b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA.

In some embodiments, the composition comprises the inactive guide RNA and the trigger nucleic acid. In some embodiments, the composition further comprises the RNA-guided endonuclease. In some embodiments, the composition further comprises a target nucleic acid. In some embodiments, the second subdomain of (a)(ii) above comprises a nucleotide sequence that is complementary to the target nucleic acid. In some embodiments, the RNA-guided endonuclease is Cas9, Cpf1 or C2c2.

In some embodiments, the first domain is located at the 5′ end of the inactive guide RNA. In some embodiments, the 3′ end of the inactive guide RNA comprises a scaffold formed by intramolecular nucleotide base pairing. In some embodiments, the unpaired subdomain (i) of the trigger nucleic acid is upstream from the unpaired subdomain (ii) of the trigger nucleic acid. In some embodiments, the first domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (ii) of the trigger nucleic acid is upstream from the unpaired subdomain (i) of the trigger nucleic acid.

In another aspect, the disclosure provides a method comprising incubating in reaction buffer in the presence of a target nucleic acid and an RNA-guided nuclease, any of the inactive guide RNAs described above and any of the trigger nucleic acids described above, to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced before or after the trigger nucleic acid.

Provided herein, in one aspect, is a composition, comprising: (a) an inactive guide ribonucleic acid (RNA) comprising a 5′ end, a 3′ end, and (i) an unpaired hairpin loop domain, (ii) a paired stem domain located adjacent to the unpaired hairpin loop domain and comprising a first subdomain contiguous with a second subdomain, and a third subdomain contiguous with a fourth guide subdomain, wherein the first subdomain and the second subdomain are respectively complementary to and bound to the third subdomain and the fourth guide subdomain, and wherein fourth guide subdomain, when not bound to the second subdomain, associates with an RNA-guided endonuclease, and (iii) an unpaired toehold domain contiguous with the first subdomain of the paired stem domain; and optionally (b) a trigger nucleic acid comprising (i) an unpaired subdomain complementary to the first subdomain of the paired stem domain of the inactive gRNA, (ii) an unpaired subdomain complementary to the second subdomain of the paired stem domain of the inactive gRNA, and (iii) an unpaired subdomain complementary to the toehold domain of the inactive gRNA.

In some embodiments, the composition comprises both the inactive guide RNA and the trigger nucleic acid. In some embodiments, the composition further comprises the RNA-guided endonuclease. In some embodiments, the composition further comprises a target nucleic acid. In some embodiments, the fourth guide subdomain of (a)(ii) above comprises a nucleotide sequence that is complementary to a target gene of interest. In some embodiments, the RNA-guided endonuclease is Cas9, Cpf1 or C2c2.

In some embodiments, the unpaired toehold domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (i) of the trigger nucleic acid is downstream from the unpaired subdomain (iii) of the trigger nucleic acid and upstream from the unpaired subdomain (ii) of the trigger nucleic acid. In some embodiments, the unpaired toehold domain is located at the 5′ end of the inactive guide RNA. In some embodiments, the 3′ end of the inactive guide RNA comprises a scaffold formed by intramolecular nucleotide base pairing. In some embodiments, the unpaired subdomain (ii) of the trigger nucleic acid is downstream from the unpaired subdomain (i) of the trigger nucleic acid and upstream from the unpaired subdomain (iii) of the trigger nucleic acid.

In another aspect, provided herein is a method comprising incubating in reaction buffer in the presence of a target nucleic acid and an RNA-guided nuclease, any of the inactive guide RNAs described above and any of the trigger nucleic acids described above, to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced before or after the trigger nucleic acid.

Provided herein, in one aspect is a composition, comprising: (a) an inactive guide ribonucleic acid (RNA) comprising a 5′ end, a 3′ end, and (i) an unpaired hairpin loop domain, and (ii) a paired stem domain located adjacent to the unpaired hairpin loop domain and comprising a first subdomain contiguous with a second subdomain, and a third subdomain contiguous with a fourth subdomain, wherein the first subdomain and the second subdomain are respectively complementary to and bound to the third subdomain and the fourth subdomain, and wherein fourth subdomain, when not bound to the second subdomain, associates with an RNA-guided endonuclease; and optionally (b) a trigger nucleic acid comprising (i) an unpaired subdomain complementary to the hairpin loop domain of the inactive gRNA, (ii) an unpaired subdomain complementary to the first subdomain of the paired stem domain of the inactive gRNA, and (iii) an unpaired subdomain complementary to the second subdomain of the paired stem domain of the inactive gRNA.

In some embodiments, the composition comprises both the inactive guide RNA and the trigger nucleic acid. In some embodiments, the composition further comprises the RNA-guided endonuclease. In some embodiments, the composition further comprises a target nucleic acid.

In some embodiments, the fourth guide subdomain of (a)(ii) above comprises a nucleotide sequence that is complementary to a target gene of interest. In some embodiments, the RNA-guided endonuclease is Cas9, Cpf1 or C2c2.

In some embodiments, the first domain is located at the 3′ end of the inactive guide RNA. In some embodiments, the unpaired hairpin loop domain of the inactive guide RNA comprises a scaffold formed by intramolecular nucleotide base pairing. In some embodiments, the unpaired subdomain (ii) of the trigger nucleic acid is downstream from the unpaired subdomain (i) of the trigger nucleic acid and upstream from the unpaired subdomain (iii) of the trigger nucleic acid. In some embodiments, the second domain is located at the 5′ end of the inactive guide RNA. In some embodiments, the 3′ end of the inactive guide RNA comprises a scaffold formed by intramolecular nucleotide base pairing. In some embodiments, the unpaired subdomain (i) of the trigger nucleic acid is downstream from the unpaired subdomain (iii) of the trigger nucleic acid and upstream from the unpaired subdomain (ii) of the trigger nucleic acid.

In another aspect, provided herein is a method comprising incubating in reaction buffer in the presence of a target nucleic acid and an RNA-guided nuclease, any of the inactive guide RNAs described above and any of the trigger nucleic acids described above, to produce an active gRNA that associates with the RNA-guided nuclease and binds to the target nucleic acid. In some embodiments, the inactive guide RNA and the trigger nucleic acid are introduced sequentially into the reaction buffer or mixture. In some embodiments, the inactive guide RNA and the trigger nucleic acid are introduced simultaneously into the reaction buffer or mixture. The inactive gRNA may be introduced before or after the trigger nucleic acid.

Provided herein, in one aspect, is a composition, comprising: (a) a supporting ribonucleic acid (RNA) strand comprising, from 5′ to 3′, a first domain, a second domain, a third domain, a fourth domain, a fifth domain, a sixth domain, a seventh domain and an eight domain, wherein the second domain is complementary to the fourth domain to form a Csy4-specific hairpin, and the sixth domain is complementary to the eighth domain to form a Cas9-specific hairpin; (b) a target RNA comprising, from 5′ to 3′, a first domain and a second domain, wherein the first domain of the target RNA is complementary to the second domain of the supporting RNA strand, and the second domain of the target RNA is complementary to the first domain of the supporting RNA strand; and (c) a guide RNA strand comprising, from 5′ to 3′, a first domain containing a guide sequence, a second domain and a third domain, wherein the first domain of the guide RNA strand associates with Cas9 nuclease, the second domain of the guide RNA is complementary to the fifth domain of the supporting RNA strand, and the third domain of the guide RNA strand is complementary to the fourth domain of the supporting RNA strand.

In some embodiments, the composition further comprises Csy4 nuclease. In some embodiments, the composition further comprises Cas9 nuclease. In some embodiments, the composition further comprises a target nucleic acid.

Provided herein, in one aspect is a composition, comprising (a) a supporting ribonucleic acid (RNA) strand comprising, from 5′ to 3′, 21 domains, wherein the 2^{nd} domain and the 3^{rd} domain are complementary to the 7^{th} domain and the 6^{th} domain, respectively, the 9^{th} domain is complementary to the 11^{th} domain, the 12^{th} domain and the 13^{th} domain are complementary to the 17^{th} domain and the 16^{th} domain, respectively, and the 19^{th} domain is complementary to the 21^{st} domain; (b) a guide RNA strand comprising, from 5′ to 3′, a 1^{st} domain, a 2^{nd} domain and a 3^{rd} domain, wherein the 1^{st} domain of the guide RNA strand associates with a RNA-guided nuclease, the 2^{nd} domain of the guide RNA strand is complementary to the 18^{th} domain of the supporting RNA strand, and the third domain of the guide RNA strand is complementary to the 17^{th} domain of the supporting RNA strand; and (c) an input RNA catalyst strand comprising, from 5′ to 3′, a 1^{st} domain, a 2^{nd} domain and a 3^{rd} domain, wherein the 1^{st} domain of the input RNA catalyst strand is complementary to the 3^{rd} domain of the supporting RNA strand, the 2^{nd} domain of the input RNA catalyst is complementary to the 2^{nd} domain of the supporting RNA strand, and the 3^{rd} domain of the input RNA catalyst is complementary to the 1^{st} domain of the supporting RNA strand.

In some embodiments, the composition further comprises the RNA-guided nuclease. In some embodiments, the composition further comprises a target nucleic acid.

Provided herein, in one aspect, is a composition, comprising: (a) a first nucleic acid complex comprising (i) a first nucleic acid strand comprising, from 5′ to 3′, a first domain, a second domain and a third domain, and (ii) a second nucleic acid strand comprising, from 5′ to 3′, a first domain, a second domain and a third domain, wherein the second domain of the second nucleic acid strand of (a)(ii) is complementary to the third domain of the first nucleic acid strand of (a)(i), and the third domain of the second nucleic acid strand of (a)(ii) is complementary to the second domain of the first nucleic acid strand of (a)(i); (b) a second nucleic acid complex comprising (i) a first nucleic acid strand comprising, from 5′ to 3′, a first domain and a second domain, each of which can associate with a DNA-guided nuclease, and (ii) a second nucleic acid strand comprising, from 5′ to 3′, a first domain and a second domain, wherein the first domain of the second nucleic acid strand of (b)(ii) is complementary second domain of the first nucleic acid strand of (a)(i), and wherein the second domain of the second nucleic acid strand of (b)(ii) is complementary to the second domain of the first nucleic acid strand of (b)(i) and is complementary to the first domain of the first nucleic acid strand of (a)(i); and (c) a nucleic acid input strand comprising, from 5′ to 3′, a first domain, a second domain and a third domain, wherein the first domain, second domain and third domain of the nucleic acid input strand are complementary to the third domain, second domain and first domain of the second nucleic acid strand of (a)(ii), respectively.

In some embodiments, the composition further comprises a DNA-guided nuclease. In some embodiments, the composition further comprises a target nucleic acid.

Provided herein, in one aspect, is a composition, comprising:(a) a first nucleic acid complex comprising (i) a first nucleic acid strand comprising, from 5′ to 3′, a first domain, a second domain, a third domain and a fourth domain, and (ii) a second nucleic acid strand comprising, from 5′ to 3′, a first domain, a second domain and a third domain, wherein the second domain of the second nucleic acid strand of (a)(ii) is complementary to the fourth domain of the first nucleic acid strand of (a)(i), and the third domain of the second nucleic acid strand of (a)(ii) is complementary to the third domain of the first nucleic acid strand of (a)(i); (b) a second nucleic acid complex comprising (i) a first nucleic acid strand comprising, from 5′ to 3′, a first domain and a second domain, each of which can associate with a DNA-guided nuclease, and (ii) a second nucleic acid strand comprising, from 5′ to 3′, a first domain and a second domain, each of which can associate with a DNA-guided nuclease (iii) a third nucleic acid strand comprising, from 5′ to 3′, a first domain, a second domain and a third domain, wherein the first domain of the third nucleic acid strand of (b)(ii) is complementary to the third domain of the first nucleic acid strand of (a)(i), wherein the second domain of the third nucleic acid strand of (b)(iii) and is complementary to the second domain of the first nucleic acid strand of (a)(i), wherein the third domain of the third nucleic acid strand of (b)(iii) is complementary to the second domain of the first nucleic acid strand of (b)(i) and is complementary to the first domain of the first nucleic acid strand of (a)(i); and (c) a nucleic acid input strand comprising, from 5′ to 3′, a first domain, a second domain and a third domain, wherein the first domain, second domain and third domain of the nucleic acid input strand are complementary to the third domain, second domain and first domain of the second nucleic acid strand of (a)(ii), respectively.

In some embodiments, the composition further comprises a DNA-guided nuclease. In some embodiments, the composition further comprises a target nucleic acid.

Provided herein, in one aspect, is a composition, comprising (a) a first nucleic acid complex comprising (i) a first nucleic acid strand comprising, from 5′ to 3′, a first domain, a second domain and a third domain, (ii) a second nucleic acid strand comprising, from 5′ to 3′, a first domain, a second domain and a third domain, and (iii) a third nucleic acid strand comprising, from 5′ to 3′, a first domain, a second domain, a third domain and a fourth domain, wherein the first domain and the second domain of the third nucleic acid strand of (a)(iii) are complementary to the first domain and the third domain of the second nucleic acid strand of (a)(ii), respectively, and wherein the third domain and the fourth domain of the third nucleic acid strand of (a)(iii) are complementary to the second and third domain of the first nucleic acid strand of (a)(i); (b) a second nucleic acid complex comprising (i) a first nucleic acid strand comprising a first domain, a second domain and a third domain, and (ii) a second nucleic acid strand comprising a domain that is complementary to the second domain of the first nucleic acid strand of (b)(i) and can associate with a DNA-guided nuclease; (c) a first nucleic acid input strand comprising, from 5′ to 3′, a first domain, a second domain and a third domain that are complementary to the third domain, second domain and first domain of the first nucleic acid strand of (a)(i), respectively; and (d) a second nucleic acid input strand comprising, from 5′ to 3′, a first domain, a second domain and a third domain that are complementary to the third domain, second domain and first domain of the first nucleic acid strand of (a)(ii), respectively.

In some embodiments, the composition further comprises a DNA-guided nuclease. In some embodiments, the composition further comprises a target nucleic acid.

Provided herein, in one aspect, is a composition, comprising: (a) a nucleic acid complex comprising (i) a first nucleic acid strand comprising, from 5′ to 3′, a first domain and a second domain that can associate with a DNA-guided nuclease, (ii) a second nucleic acid strand comprising, from 5′ to 3′, a first domain and a second domain, and (iii) a third nucleic acid strand comprising, from 5′ to 3′, a first domain, a second domain, a third domain and a fourth domain, wherein the first domain of the third nucleic acid strand of (a)(iii) is complementary to the first domain of the second nucleic acid strand of (a)(ii), and wherein the third domain of the third nucleic acid strand of (a)(iii) is complementary to the first domain of the first nucleic acid strand of (a)(i); and (b) a first nucleic acid input strand comprising, from 5′ to 3′, a first domain and a second domain, or a second nucleic acid strand comprising, from 5′ to 3′, a first domain and a second domain, wherein the first domain and the second domain of the first nucleic acid input strand of (b) are complementary to the fourth domain and the third domain of the third nucleic acid strand of (a)(iii), respectively, and wherein the first domain and the second domain of the second nucleic acid input strand of (b) are complementary to the second domain and the first domain of the third nucleic acid strand of (a)(iii), respectively.

In some embodiments, the composition further comprises a DNA-guided nuclease. In some embodiments, the composition further comprises a target nucleic acid.

Provided herein, in one aspect, is a cell comprising a nucleic acid encoding any of the inactive guide RNAs described herein. In another aspect, the disclosure provides a cell comprising any of the inactive guide RNAs described herein. In some embodiments, the cell further comprises any of the trigger nucleic acids (e.g., cognate trigger nucleic acids) described herein.

Provided herein, in one aspect, is a vector comprising a nucleic acid encoding any of the inactive guide RNAs described herein. Another aspect of the present disclosure provides a vector comprising a nucleic acid encoding any of the trigger nucleic acids described herein. In some embodiments, the vector comprises a nucleic acid that encodes any of the inactive guide RNAs and any of their cognate trigger nucleic acids described herein.

Provided herein, in one aspect, is a nucleic acid molecule encoding any of the inactive guide RNAs described herein. Another aspect of the present disclosure provides a nucleic acid molecule encoding any of the trigger nucleic acids (e.g., cognate trigger nucleic acids) described herein. In some embodiments, the nucleic acid molecule encodes any of the inactive guide RNAs and any of their cognate trigger nucleic acids described herein.

In some embodiments, the inactive gRNA and trigger nucleic acid are present in the same nucleic acid or vector. In other embodiments, the inactive gRNA and trigger nucleic acid are present in separate nucleic acids or vectors.

Provided herein, in one aspect, is a method of using any one of the foregoing compositions to modify genomic nucleic acid in a cell.

In another aspect, provided herein is a cell comprising any one of the foregoing compositions. In some embodiments, the cell is a prokaryotic cell or a eukaryotic cell.

Also provided herein, in one aspect, are kits or compositions comprising (a) any of the inactive guide RNAs described herein; and/or (b) any of their cognate trigger nucleic acids described herein, and optionally (c) an RNA-guided nuclease.

### BRIEF DESCRIPTION OF DRAWINGS

FIG. 1 shows a Cas9 guide RNAs in complex with target DNA. The sequences, from top to bottom, correspond to SEQ ID NOs: 26-29 (two-component guide RNA) and SEQ ID NOs: 26, 27, and 30 (single guide RNA).

FIG. 2 shows a Cpf1 guide RNA in complex with target DNA. The sequences, from top to bottom, correspond to SEQ ID NOs: 31-33.

FIG. 3 shows a C2c2 guide RNA in complex with target RNA. The sequences, from top to bottom, correspond to SEQ ID NOs: 34 and 35.

FIG. 4 shows a general mechanism for toehold switch-gRNA systems.

FIGS. 5A-5B show general constructs and general mechanisms for toehold switch-gRNA systems. “Domain 1” represents a region of the guide RNA (gRNA).

FIG. 6 shows an example of a user-defined ˜20 nucleotide guide sequences that defines the DNA sequences targeted by a Cas9 complex. The scaffold sequence is necessary for Cas9-binding. Generic sequence of sgRNA (used throughout the Examples, unless otherwise specified): 5′NNNNNNNNNNNNNNNNNNNNGUUUUAGAGCUAGAAAUAG CAAGUUAAAAUAAGGCUAGUCCGUUAUCAACUUGAAAAAGUGGCACCGAGUCGGU GC 3′ (SEQ ID NO: 1) (20-nt guide sequence in bold, 76-nt standard scaffold sequence in italic).

FIGS. 7A-7D show an example of a switch-gRNA design wherein the guide sequence is fully sequestered by the switch. Domain a* is the masking domain. By masking the guide sequence, domain a* inactivates the guide RNA. Domain x (FIG. 7A) is a toehold domain. This toehold domain enables toehold strand-mediated displacement by single-stranded nucleic acid triggers. Domains w and y are a linker domains. Domains w and y can be shortened or removed without altering the system. Further, domains w and y can be used as a toehold domain similar to domain x. For example, in FIG. 7B, domain y is a toehold domain. FIGS. 7C and 7D show further embodiments of FIGS. 7A and 7B, respectively, wherein the trigger (domains a and b1) is complementary to the guide domain and at least one nucleotide from the scaffold domain (domains a* and b1*, respectively).

FIGS. 8A-8D show another example of a switch-gRNA design wherein the guide sequence is fully sequestered by the switch.

FIGS. 9A-9B show an example of a switch-gRNA design wherein the guide sequence is adjacent to the switch. Domain z* is the masking domain. Domain z* is not complementary to the guide sequence.

FIGS. 10A-10B show another example of a switch-gRNA design wherein the guide sequence is adjacent to the switch. Domain z* is the masking domain. Domain z* is not complementary to the guide sequence. Unexpectedly, results from cleavage assays show that the presence of the z-z* duplex adjacent to the guide sequence is sufficient to inactivate the guide RNA.

FIGS. 11A-11B show an example of a switch-gRNA design wherein the guide sequence is partially sequestered by the switch.

FIGS. 12A-12B show another example of a switch-gRNA design wherein the guide sequence is partially sequestered by the switch.

FIG. 13A-13B shows switch-gRNA designs where any of domain ‘a,’ ‘b,’ ‘c’ or ‘d’ of the two-component guide RNA or any of domain ‘a’ or ‘b’ of the single-guide RNA are sequestered by the switch.

FIG. 14 shows an example of a general mechanism for Csy4-mediated release

FIG. 15 shows an example of a general mechanism for catalytic circuit

FIG. 16 shows an example of guide DNA release in the presence of an RNA input sequence (single input, single output).

FIG. 17 shows an example of multiple guide DNA release in the presence of RNA input sequences (single input, multiple output).

FIG. 18 shows release of guide DNA in the presence of RNA input sequence 1 AND RNA input sequence 2 (multiple input, single output).

FIG. 19 shows release of guide DNA in the presence of RNA input sequence 1 OR RNA input sequence 2 (multiple input, single output).

FIG. 20 shows results from a cleavage assay using a switch-gRNA configured as shown in FIG. 7A. This switch-gRNA is inactive in the absence of a trigger (the target DNA is not cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4).

FIG. 21 shows results from a cleavage assay using a switch-gRNA configured as shown in FIG. 7A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the x* domain is 14 nucleotides (GUCAUACUCCAAAG; SEQ ID NO: 5), and the length of the a domain is 16 nucleotides (GAUUUCUUCUUGCGCU; SEQ ID NO: 6).

FIG. 22 shows results from a cleavage assay using a switch-gRNA configured as shown in FIG. 7A, in the presence of DNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the x* domain is 14 nucleotides (GTCATACTCCAAAG; SEQ ID NO: 7), and the length of the a domain is 16 nucleotides (GATTTCTTCTTGCGCT; SEQ ID NO: 8).

FIG. 23 shows results from a cleavage assay using a switch-gRNA configured as shown in FIG. 7A, in the presence of a RNA trigger x*a having a length shorter than the RNA trigger used for the data shown in FIG. 21. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the x* domain is 14 nucleotides (GUCAUACUCCAAAG; SEQ ID NO: 5), and the length of the a domain is 8 nucleotides (GAUUUCUU) or 12 nucleotides (GAUUUCUUCUUG; SEQ ID NO: 10).

FIG. 24 shows results from a cleavage assay using a switch-gRNA configured as shown in FIG. 7A, in the presence of a RNA trigger aw*. This switch-gRNA is activated in the presence of RNA trigger aw* (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the w domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the a domain is 16 nucleotides (GAUUUCUUCUUGCGCU; SEQ ID NO: 6), and the length of the w* domain is 10 nucleotides (GGGAUGUAUU; SEQ ID NO: 11).

FIG. 25 shows results from a cleavage assay using a switch-gRNA configured as shown in FIG. 7A, in the presence of a two RNA triggers: A1-u x*2 a; and A2-x*12 u*. This switch-gRNA is activated only in the presence of RNA triggers A1 and A2 (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (AAUACAUCCC; SEQ ID NO: 2); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the u domain is 16 nucleotides (AUAACUAAGAACGACGAUGACACA; SEQ ID NO: 12); the length of the x* domain is 2 nucleotides (AG); the length of the a domain is 16 nucleotides (GAUUUCUUCUUGCGCU; SEQ ID NO: 6); the length of the x* domain is 12 nucleotides (GUCAUACUCCAA; SEQ ID NO: 36); and the length of the u* domain is 16 nucleotides (ACAGUCAUCGUCGUUCUUAGUUAU; SEQ ID NO: 13).

FIG. 26 shows results from a cleavage assay using a switch-gRNA configured as shown in FIG. 7A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 2); the length of the scaffold is 76 nucleotides (standard); the length of the y domain is 10 nucleotides (AAUACAUCCC; SEQ ID NO: 2); the length of the a* domain is 16 nucleotides (UGACCGACUGUGAACC; SEQ ID NO: 14); and the length of the x domain is 14 nucleotides (CUUUGGAGUAUGAC; SEQ ID NO: 4). The length of the x* domain is 14 nucleotides (GUCAUACUCCAAAG; SEQ ID NO: 5), and the length of the a domain is 16 nucleotides (GGUUCACAGUCGGUCA; SEQ ID NO: 15).

FIG. 27 shows results from a cleavage assay using a switch-gRNA configured as shown in FIG. 7A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the w domain is varied: 10 nucleotides (AAUACAUCCC; SEQ ID NO: 2), 5 nucleotides (AAAGA), or zero nucleotides; the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the x* domain is 14 nucleotides (GUCAUACUCCAAAG; SEQ ID NO: 5), and the length of the a domain is 16 nucleotides (GAUUUCUUCUUGCGCU; SEQ ID NO: 6).

FIG. 28 shows results from a cleavage assay using a switch-gRNA configured as shown in FIG. 7A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 55 nucleotides (GUUUUAGAGCUAUGCUGAAAAGCAUAGCAAGUUAAAAUAAGGCUAGUCCGUU AUC; SEQ ID NO: 16); the length of the a* domain is 16 nucleotides (AGCGCAAGAAGAAAUC; SEQ ID NO: 3); and the length of the x domain is 14 nucleotides (CUUUGGAGUAUGAC; SEQ ID NO: 4). The length of the x* domain is 14 nucleotides (GUCAUACUCCAAAG; SEQ ID NO: 5), and the length of the a domain is 16 nucleotides (GAUUUCUUCUUGCGCU; SEQ ID NO: 6).

FIG. 29 shows results from a cleavage assay using a switch-gRNA configured as shown in FIG. 9A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 20 nucleotides (targeting protospacer 1); the length of the scaffold is 76 nucleotides (standard); the length of the y domain is 10 nucleotides (GCCUACUCAU; SEQ ID NO: 17); the length of the a* domain is 16 nucleotides (GAGUUGUAAUUGUGCC; SEQ ID NO: 18); and the length of the x domain is 14 nucleotides (UUGUAUAUGUGUCC; SEQ ID NO: 19). The length of the x* domain is 13 nucleotides (GGACACAUAUACA; SEQ ID NO: 20), and the length of the a domain is 16 nucleotides (GGCACAAUUACAACUC; SEQ ID NO: 21).

FIG. 30 shows results from a cleavage assay using a switch-gRNA configured as shown in FIG. 10A, in the presence of RNA trigger x*a. This switch-gRNA is activated in the presence of RNA trigger x*a (the target DNA is cleaved). The cleavage assay was performed with a purified CRISPR/Cas9 protein (commercially available from PNA bio). The target DNA template is a synthetic dsDNA (500 nucleotides long) comprising protospacer 1 and 2 sequences. A similar methodology may be used for the methods provided herein, although the disclosure is not so limited. The length of the guide domain is 10 nucleotides (targeting protospacer 1); the length of the scaffold is 76 nucleotides (standard); the length of the y domain is 6 nucleotides (AAUUCC); the length of the a* domain is 16 nucleotides (GGCACCUAACAUACAC; SEQ ID NO: 22); the length of the a domain (of the switch-gRNA) is 16 nucleotides (GUGUAUGUUAGGUGCC; SEQ ID NO: 23); and the length of the x domain is 14 nucleotides (GGUUAGUAAUGUUA; SEQ ID NO: 24). The length of the x* domain is 14 nucleotides (UAACAUUACUAACC; SEQ ID NO: 25), and the length of the a domain (of the trigger) is 16 nucleotides (GGCACCUAACAUACAC; SEQ ID NO: 22).

FIG. 31 shows an overview of the switch-gRNAs that enable the control of CRISPR genome editing and genome regulation functions by endogenous RNA sequences (top), and an example of in vivo sensing and monitoring of endogenous RNA using a series of orthogonal switch-gRNAs and different output signals (bottom).

FIG. 32 provides an example of switchable-guide RNA and shows the design and overall mechanism.

FIG. 33 presents an in vitro binding assay (band shift assay or electrophoretic mobility shift assay) using a switch-gRNA configured as shown in FIG. 7A, in the presence of RNA trigger x*a. The binding assay was performed with an engineered nuclease-deficient Cas9, termed dCas9 (commercially available from PNA bio). The synthetic template dsDNA (50-nt long) contained the proto spacer 1 sequence and was fluorescently labeled to visualize the band shift. This switch-gRNA is activated in the presence of RNA trigger x*a, as the dCas9 complex binds to the template DNA resulting in a band shift on the gel.

### DESCRIPTION

RNA-guided endonucleases such as the CRISPR effectors Cas9, Cpf1, C2c2 use guide RNA molecules (gRNAs) to recognize and cleave complementary target nucleic acids. Cas9 and Cpf1 cleave DNA molecules while C2c2 cleaves RNA molecules. Provided herein, in some embodiments, are engineered gRNAs that are ‘activated’ (capable of binding to an effector nuclease) by specific RNA or DNA sequences. This allows controlling the activity of the aforementioned nucleases, including their mutated forms. The present disclosure also provides, in some embodiments, engineered gDNAs that can be activated by specific DNA sequences.

### Conditional Activation of RNA-Guided Nucleases Using Cas9, Cpf1 or C2c2

An “RNA-guided nuclease” is a programmable endonuclease that can be used to perform targeted genome editing. The programmable nature of an RNA-guided nuclease, such as Cas9, Cpf1 or C2c2, is a result of its association with a guide RNA (gRNA) that uses ˜20 variable nucleotides at its 5′ end to base pair with (are complementary to) a target DNA sequence cleaved by the nuclease. In some embodiments, the RNA-guided nuclease is Cas9. In some embodiments, the RNA-guided nuclease is Cpf1. In some embodiments, the RNA-guided nuclease is C2c2. Other RNA-guided nucleases are encompassed by the present disclosure.

Cas9 (CRISPR associated protein 9) is an RNA-guided nuclease of a class 2 CRISPR (Clustered Regularly Interspersed Palindromic Repeats) adaptive immunity system in Streptococcus pyogenes, among other bacteria. CRISPR systems for editing, regulating and targeting genomes may comprise at least two distinct components: (1) a guide RNA (gRNA) and (2) Cas9. A gRNA is a single chimeric transcript that combines the targeting specificity of endogenous bacterial CRISPR targeting RNA (crRNA) with the scaffolding properties of trans-activating crRNA (tracrRNA). Typically, a gRNA used for genome editing is transcribed from either a plasmid or a genomic locus within a cell. The gRNA transcript forms a complex with Cas9 (or other RNA-guided nuclease), and then the gRNA/Cas9 complex is recruited to a target sequence as a result of the base-pairing between the crRNA sequence and its complementary target sequence in genomic DNA, for example.

In a typical synthetic CRISPR/Cas9 genome editing system, a genomic sequence of interest (genomic target sequence) is modified by use of a gRNA complementary to the sequence of interest, which directs the gRNA/Cas9 complex to the target (Sander J D et al., 2014 Nature Biotechnology 32, 247-355, incorporated by reference herein). The Cas9 endonuclease cuts the genomic target DNA upstream of a protospacer adjacent motif (PAM), resulting in double-strand breaks. Repair of the double-strand breaks often results in inserts or deletions at the double-strand break site. Using an engineered nuclease-deficient Cas9, termed dCas9, enables the repurposing of the system for targeting genomic DNA without cleaving it, thereby enabling transcription regulation when fused to transcription activators (e.g., Cas9-VP64) or repressors (Cas9-KRAB). CRISPR-Cas target sequence specificity is readily programmable and is specified by 20 nt sequence on the sgRNA complementary to the desired DNA target sequence.

Cpf1 is also a RNA-guided nuclease of a class 2 CRISPR-Cas system (Zetsche et al., 2015, Cell 163: 1-13, incorporated by reference herein). Cpf1, like Cas9, is a two-component RNA programmable DNA nuclease. Targeted DNA is cleaved as a 5-nt staggered cut distal to a 5′ T-rich protospacer adjacent motif (PAM). There are two Cpf1 orthologs that exhibit robust nuclease activity in human cells, either of which may be used as provided herein. Enzymes that are functionally similar to Cpf1 may be used in accordance with the present disclosure.

C2c2 is a class 2 type VI-A CRISPR-Cas effector from the bacterium Leptotrichia shahii and provides interference against RNA phage. C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins.

While CRISPR sequence specificity can be customized easily, the ability to control where and when it is active is currently limited. Native CRISPR-Cas systems only implement the logic “see sequence A, cut/bind sequence A”, and previous efforts to apply conditions to this logic focused mostly on control of Cas proteins. This type of control cannot be multiplexed, is not easily programmable, and is limited to sensing specific exogenous small molecules or light pulses. Engineering sgRNAs to implement conditional logic, as provided herein, allows for programmable sensing of endogenous or synthetic RNA components using logic such as “see sequence A, cut/bind sequence B”, as well as more complex logic operations such as “see sequence A AND B, cut/bind sequence C” (2-Input AND gate). By modulating CRISPR activity at the sgRNA level and not at the protein level, this strategy enables multiplexed detection by expressing multiple RNA sensors in each cell.

A “guide” RNA (gRNA), as indicated above, is a short synthetic RNA composed of a scaffold sequence necessary for RNA-guided nuclease (e.g., Cas9) binding and a user-defined ˜20 (e.g., 20±5 or 20±10) nucleotide “spacer” or “targeting” sequence that defines the target (e.g., genomic target) to be modified. Thus, one can change the (genomic) target of an RNA-guided nuclease (e.g., Cas9) by simply changing the targeting sequence present in the gRNA. In some embodiments, a gRNA has a length of 10 to 100 nucleotides. For example, a gRNA may have a length of 10-90, 10-80, 10-70, 10-60, 10-50, 10-40, 10-35, 10-30, 10-25, 10-20, 10-15, 15-100, 15-90, 15-80, 15-70, 15-60, 15-50, 15-40, 15-35, 15-30, 15-25, 15-20, 20-100, 20-90, 20-80, 20-70, 20-60, 20-50, 20-40, 20-35, 20-30 or 20-25 nucleotides. In some embodiments, a gRNA has a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Longer gRNAs are encompassed by the present disclosure.

Engineered Guide RNAs for Cas9

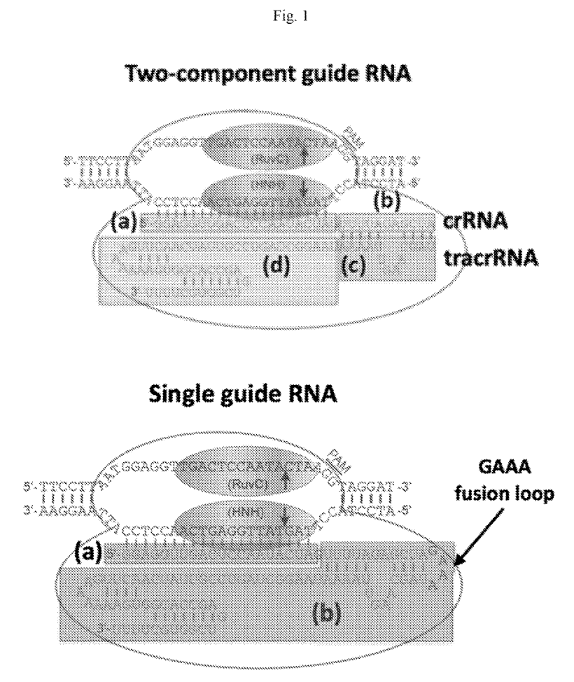

There are at least two different guide RNA systems that can be used with Cas9. The first system (FIG. 1, top) is a native system originally discovered in Streptococcus pyogenes. It includes two distinct RNA molecules referred to as tracrRNA and crRNA. Both RNA molecules are used to activate Cas9. The second system (FIG. 1, bottom) is a synthetic system referred to as single guide RNA (sgRNA). The single guide RNA was created by truncating and fusing tracrRNA with crRNA using a short linker sequence (e.g., GAAA).

In some embodiments, where a two-component guide system is used, the approximate length of domain ‘a’ (also referred to as protospacer) is 20 nucleotides (+/−10 nt), the approximate length of domain ‘b’ is 10 nucleotides (nt) (+/−10 nt), the approximate length of domain ‘c’ is 10 nucleotides (+/−10 nt), and the approximate length of domain ‘d’ is 20 nucleotides (+/−10 nt). Thus, in some embodiments, domain ‘a’ has a length of 10-30 nucleotide (e.g., 10, 15, 20, 25 or 30 nucleotides). In some embodiments, domain ‘a’ has a length of 23, 24 or 25 nucleotides. In some embodiments, domain ‘b’ has a length of 10-30 nucleotide (e.g., 10, 15, 20, 25 or 30 nucleotides). In some embodiments, domain ‘b’ has a length of 23, 24 or 25 nucleotides. In some embodiments, domain ‘c’ has a length of 10-30 nucleotide (e.g., 10, 15, 20, 25 or 30 nucleotides). In some embodiments, domain ‘c’ has a length of 23, 24 or 25 nucleotides. In some embodiments, domain ‘c’ forms a hairpin loop secondary structure, as depicted in FIG. 1, for example. In some embodiments, domain ‘d’ has a length of 10-30 nucleotide (e.g., 10, 15, 20, 25 or 30 nucleotides). In some embodiments, domain ‘d’ has a length of 23, 24 or 25 nucleotides. In some embodiments, domain ‘d’ forms at least one hairpin loop secondary structure, as depicted in FIG. 1 (top), for example.

In some embodiments, wherein a single-guide RNA system is used, the approximate length of domain ‘a’ (also referred to as protospacer) is 20 nucleotides (+/−10 nt), and the approximate length of domain ‘b’ is 40-90 nucleotides (+/−10 nt). Thus, in some embodiments, domain ‘a’ has a length of 10-30 nucleotide (e.g., 10, 15, 20, 25 or 30 nucleotides). In some embodiments, domain ‘b’ has a length of 30, 40, 50, 60, 70, 80, 90 or 100 nucleotides.

Engineered Guide RNAs for Cpf1

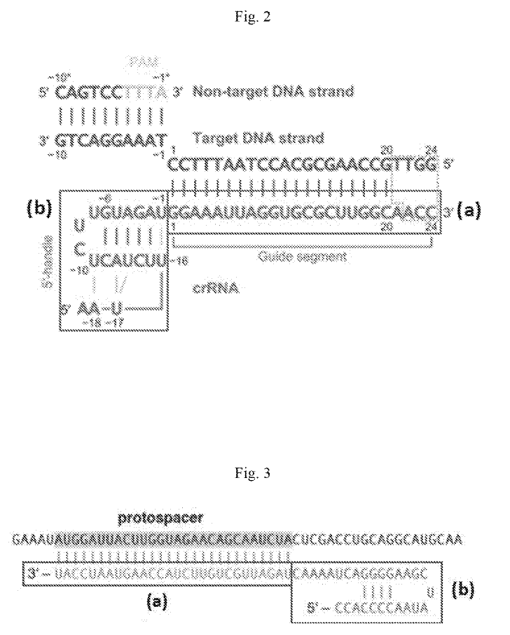

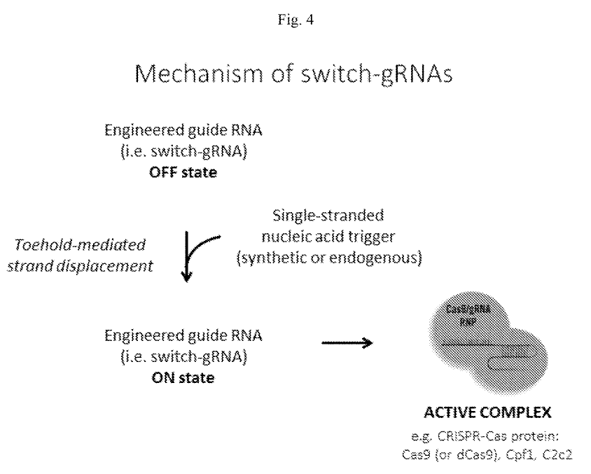

CRISPR-Cpf1 is a class 2 CRISPR system RNA-guided endonucleases discovered in Prevotella and Francisella bacteria. In contrast to Cas9, Cpf1 uses only a single guide RNA. Methods and constructs presented in FIG. 1, relating to Cas9, also apply for Cpf1 guide RNA. Cpf1 guide RNA can be divided in two domains (domain ‘a’ and ‘b’) (FIG. 2) and switches can be incorporated in these domains following the principles described relating to Cas9.

In some embodiments, the approximate length of domain ‘a’ (also referred to as protospacer) is 24 nucleotides (+/−10 nt), and the approximate length of domain ‘b’ is 20 nucleotides (+/−10 nt). Thus, in some embodiments, domain ‘a’ has a length of 14 to 34 nucleotide (e.g., 14, 15, 16, 17, 18, 19, 20, 21, 22, 23, 24, 25, 26, 27, 28, 29, 30, 31, 32, 33 or 34 nucleotides). In some embodiments, domain ‘b’ has a length of 10 to 30 nucleotides (e.g., 10, 11, 12, 13, 14, 15, 16, 17, 18, 19, 20, 21, 22, 23, 24, 25, 26, 27, 28, 29 or 30 nucleotides). In some embodiments, domain ‘b’ forms at least one hairpin loop secondary structure, as depicted in FIG. 2, for example.

Engineered Guide RNAs for C2c2

C2c2 from the bacterium Leptotrichia shahii is a RNA-guided RNase that can be efficiently programmed to degrade any ssRNA by specifying a 28-nt sequence on the guide RNA. In contrast to Cas9, C2c2 uses only a single guide RNA.

Methods and constructs presented in FIG. 1, relating to Cas9, also apply for C2c2 guide RNA. C2c2 guide RNA can be divided in two domains (domain ‘a’ and ‘b’) (FIG. 3) and switches can be incorporated in these domains following the principles relating to Cas9.

In some embodiments, the approximate length of domain ‘a’ (also referred to as protospacer) is 30 nucleotides (+/−15 nt), and the approximate length of domain ‘b’ is 20 nucleotides (+/−10 nt). Thus, in some embodiments, domain ‘a’ has a length of 15 to 45 nucleotide (e.g., 15, 20, 25, 30, 35, 40 or 45 nucleotides). In some embodiments, domain ‘a’ has a length of 28 nucleotides. In some embodiments, domain ‘b’ has a length of 10 to 30 nucleotides (e.g., 10, 11, 12, 13, 14, 15, 16, 17, 18, 19, 20, 21, 22, 23, 24, 25, 26, 27, 28, 29 or 30 nucleotides). In some embodiments, domain ‘b’ forms at least one hairpin loop secondary structure, as depicted in FIG. 3, for example.

Toehold Switch Systems

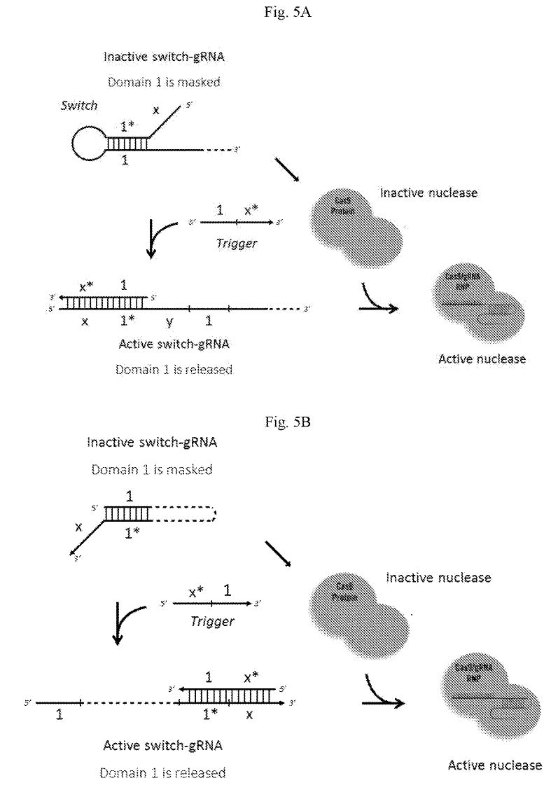

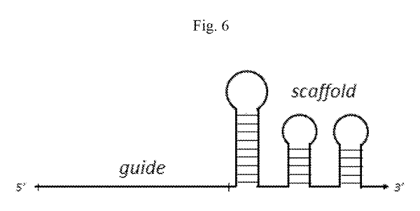

Inactive gRNAs were engineered by inserting a toehold switch motif (referred to as a “switch”) in a gRNA to sequester regions that are required for interaction with Cas9 and/or activation of the nuclease activity (illustrated by domain 1 in FIGS. 5A and 5B). In FIGS. 5A and 5B, domain 1 binds to (is complementary to) guide domain 1* of the switch-guide RNA. The trigger nucleic acid functions to dissociate the binding of domain 1 to guide domain 1* such that guide domain 1* is free to bind to Cas9 or another effector/gene editing nuclease.

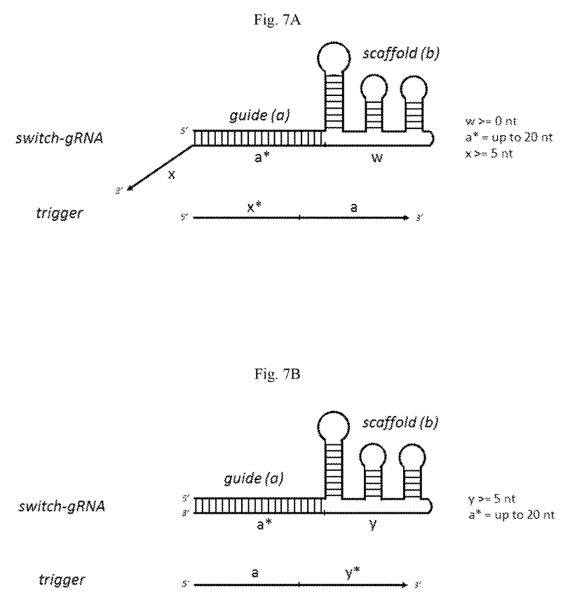

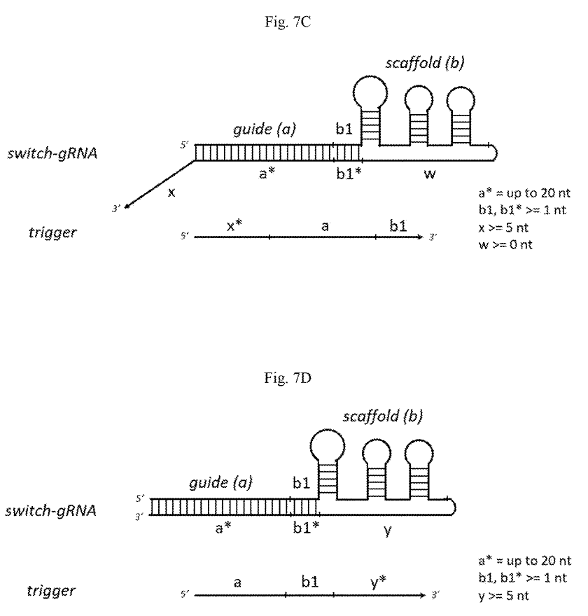

In some embodiments, the activation of gRNAs is triggered by a mechanism referred to as toehold-mediated strand displacement (Zhang D Y & Seelig G, Nature Chemistry 3, 103-113 (2011), incorporated herein by reference). When a specific single-stranded RNA sequence is present (referred to as a “trigger”) such as an endogenous RNA (e.g., mRNA or non-coding RNA) or a synthetic RNA, the trigger binds to the inactive gRNA and induces a conformational change that releases the sequestered regions, thereby promoting activation of the gRNA and formation of the active Cas9 complex (see, e.g., FIGS. 7A, 7C, 8A, 8C, 9A, 10A, 11A and 12A). Single-stranded DNA molecules can also be used as triggers.

In the presence of a trigger RNA (or trigger DNA), the engineered gRNA transitions from an inactivate state to an active state which enables the formation of the active Cas9 complex. This system can be programmed to detect any single-stranded RNA or DNA triggers.

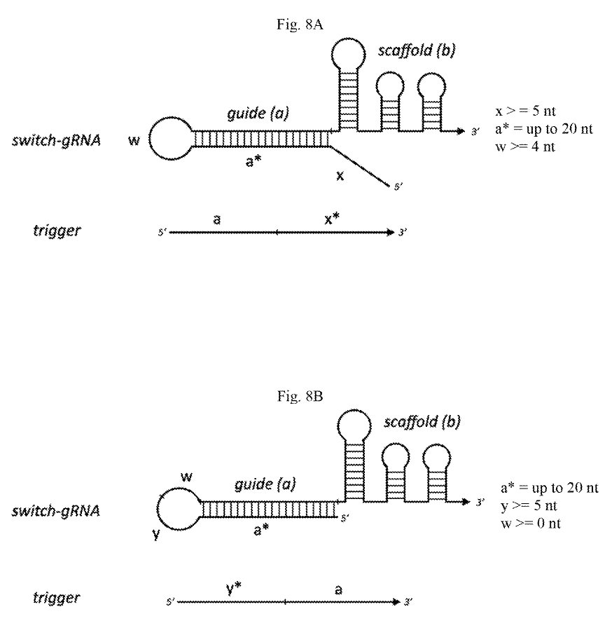

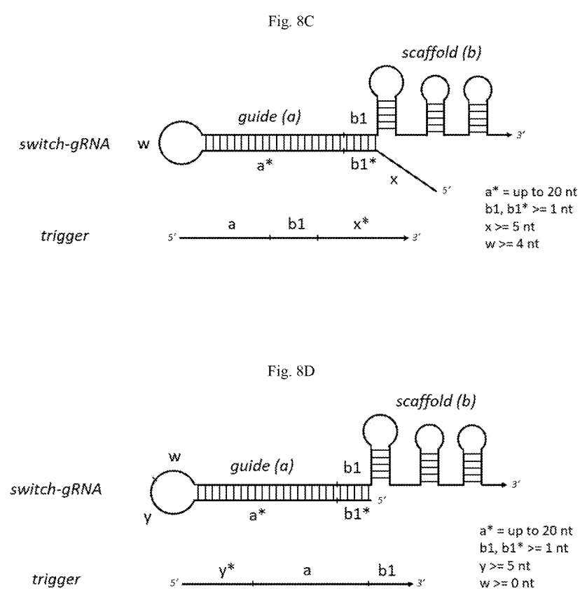

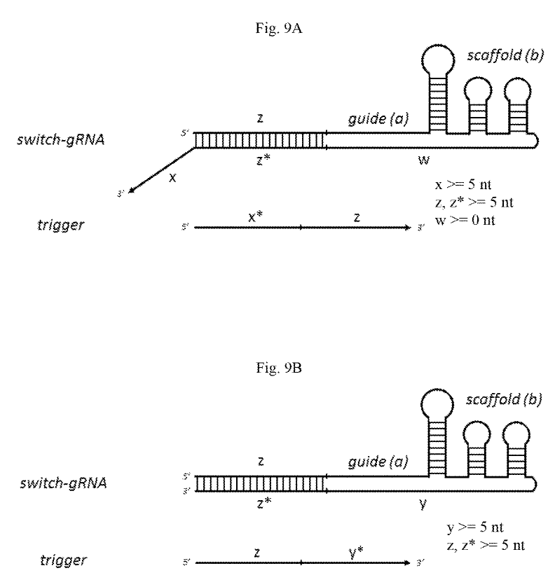

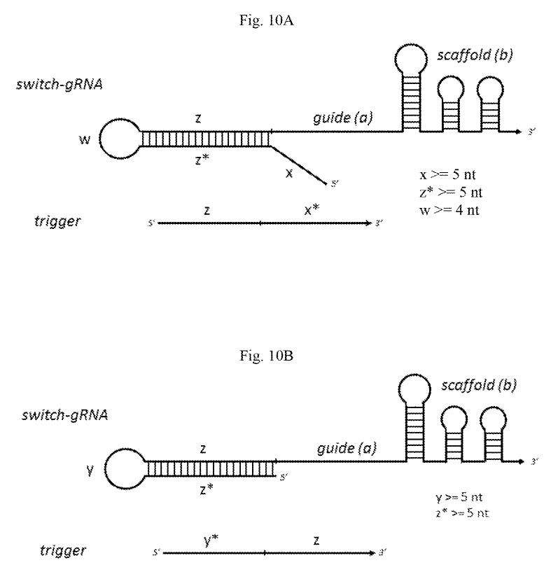

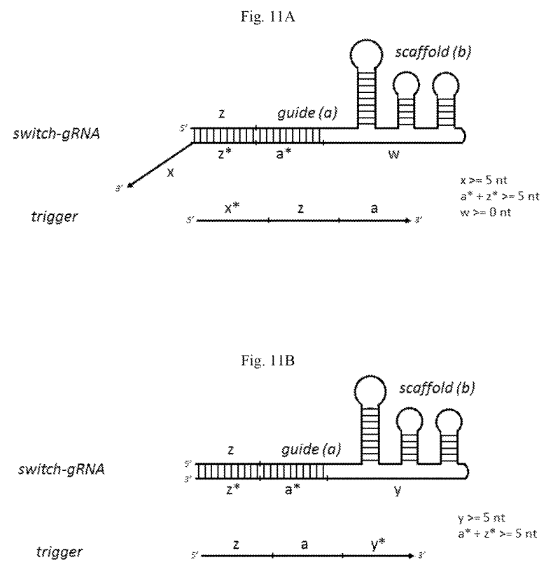

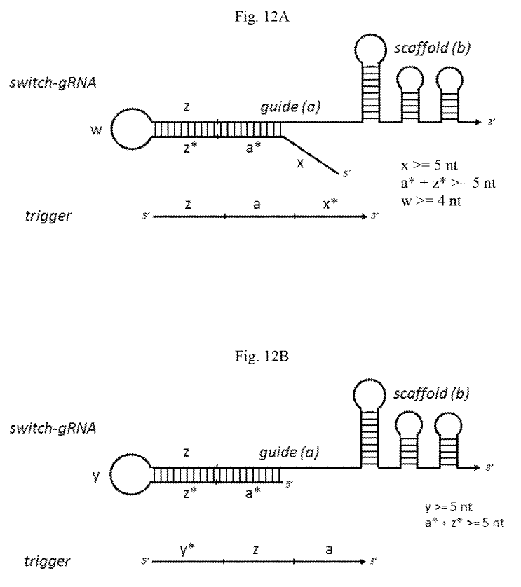

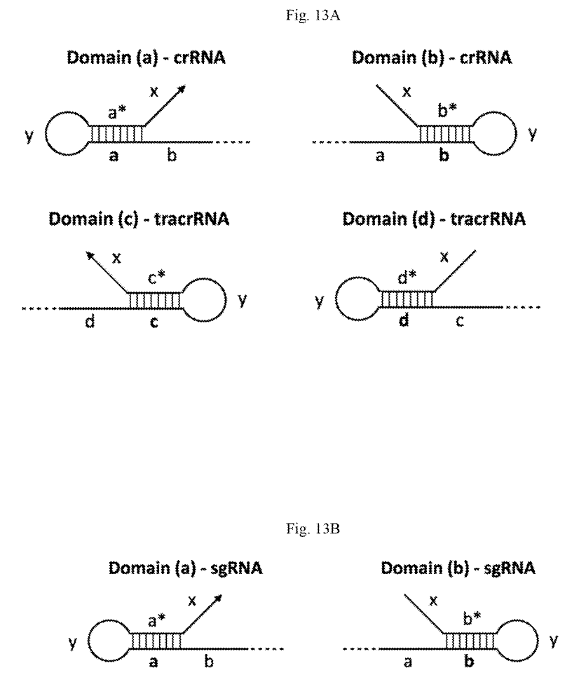

Based on this technology, several embodiments have been developed and described below. All examples are given for guide domain ‘a’ of the guide RNA (FIG. 1). Switch-guide RNAs in which the guide domain ‘a’ is fully sequestered are depicted in FIGS. 7A-8D. Switch-guide RNAs in which a switch is located adjacent to (e.g., immediately adjacent to, without intervening nucleotides) the guide domain ‘a’ are depicted in FIGS. 9A-10B. The presence of this adjacent switch is sufficient to alter complex formation and/or nuclease activity. The switch-guide RNAs in which the guide domain ‘a’ is partially sequestered are depicted in FIGS. 11A-12B.

The switch can be activated using at least two different mechanisms. One mechanism (FIGS. 7A, 7C, 8A, 8C, 9A, 10A, 11A, and 12A) is a toehold-mediated strand displacement mechanism, where the switch is opened from the bottom of the switch (domain ‘x’). The interaction between domain ‘x’ of the switch and domain ‘x*’ of the trigger is sufficient to prime the strand displacement reaction and therefore open the switch and activate the gRNA. Another mechanism (FIGS. 7B, 7D, 8B, 8D, 9B, 10B, 11B, and 12B) is a mechanism where a switch is opened from a loop (domain ‘y’). The interaction between domain ‘y’ of the switch and domain ‘y*’ of the trigger is sufficient to prime the strand displacement reaction and therefore open the switch and activate the gRNA.

The example switch-gRNAs shown in FIGS. 7A, 7C, 8A, 8C, 9A, 10A, 11A, and 12A include toehold domain ‘x’, guide domain ‘a’, domain ‘a*” complementary to guide domain ‘a’, and linker domain ‘w’ or ‘y’. The example switch-gRNAs shown in FIGS. 9A-12B also include domain ‘z’, located adjacent to guide domain ‘a’. Switch-gRNAs also include a scaffold domain, either within linker domain ‘w’ (e.g., FIGS. 7A and 7C) or ‘y’ (e.g., FIGS. 7B and 7D) or located at the 3′ end of the switch-gRNA (e.g., FIGS. 8A-8D). In addition, switch-gRNAs may also include a ‘b1’ domain located at the 5′ end of the guide domain ‘a’. For two-component guide RNAs, the scaffold may include components (b), (c) and (d) of crRNA/tracrRNA, as depicted in FIG. 1 (top). For single guide RNAs, the scaffold may include component (b), as depicted in FIG. 1 (bottom). Any sequence composition and/or length can be used for domain ‘x’, ‘y’, or ‘w’; these domains are not constrained by the sequence of the gRNA.

Domain ‘x’ refers to an unpaired nucleotide (e.g., RNA) domain located at the 3′ end of a switch-gRNA (see, e.g., FIGS. 7A and 7C) or located at the 5′ end of a switch-gRNA (see, e.g., FIGS. 8A and 8C). In some embodiments, the length of domain ‘x’ is 10 (e.g., contiguous) nucleotides (+/−10 nt). In some embodiments, the length of domain ‘x’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘x’ is 5, 6, 7, 8, 9, 10, 11, 12, 13, 14, 15, 16, 17, 18, 19, or 20 nucleotides.

In some embodiments, the length of domain ‘y’ (not including the gRNA scaffold sequences) is 5 (e.g., contiguous) nucleotides (+/−10 nt). In some embodiments, the length of domain ‘y’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘y’ is 5, 6, 7, 8, 9, 10, 11, 12, 13, 14, 15, 16, 17, 18, 19, or 20 nucleotides.

In some embodiments, the length of domain ‘w’ (not including the gRNA scaffold sequences) is 4 (e.g., contiguous) nucleotides (+/−10 nt). In some embodiments, the length of domain ‘w’ (not including the gRNA scaffold sequences) is 5 (e.g., contiguous) nucleotides (+/−10 nt). In some embodiments, the length of domain ‘w’ (or ‘w’) is 1-100 nucleotides, or 5-100 nucleotides (e.g., 1-10, 1-20, 1-30, 1-40, 1-50, 1-60, 1-70, 1-80, 1-90, 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘w’ is 1, 2, 3, 4, 5, 6, 7, 8, 9, 10, 11, 12, 13, 14, 15, 16, 17, 18, 19, or 20 nucleotides. In some embodiments, the domain ‘w’ is absent.

In some embodiments, the length of domain ‘a’ (the guide domain) is 10 (e.g., contiguous) nucleotides (+/−10 nt). In some embodiments, the length of domain ‘a’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘a’ is 10, 11, 12, 13, 14, 15, 16, 17, 18, 19, 20, 21, 22, 23, 24, 25, 26, 27, 28, 29, or 30 nucleotides.

In some embodiments, the length of domain ‘z’ is 20 (e.g., contiguous) nucleotides (+/−10 nt). In some embodiments, the length of domain ‘z’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘z’ is 5, 6, 7, 8, 9, 10, 11, 12, 13, 14, 15, 16, 17, 18, 19, or 20 nucleotides.

In some embodiments, the length of domain ‘b1’ may be 5 (e.g., contiguous) nucleotides (+/−10 nt). In some embodiments, the length of domain ‘b1’ is 1-100 nucleotides or 5-100 nucleotides (e.g., 1-10, 1-20, 1-30, 1-40, 1-50, 1-60, 1-70, 1-80, 1-90, 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘b1’ is 1, 2, 3, 4, 5, 6, 7, 8, 9, 10, 11, 12, 13, 14, 15, 16, 17, 18, 19, or 20 nucleotides. Domain ‘b1’ may be located at the 3′ end of the trigger nucleic acid or it may be located elsewhere on the trigger nucleic acid, 3′ and adjacent to domain ‘a’. In some embodiments, domain ‘b1’ may be absent.

In some embodiments, the length of the scaffold is 50-200 nucleotides (e.g., 50-60, 50-70, 50-80, 50-90, 50-100, or 50-150 nucleotides). In some embodiments, the length of the scaffold is 60-80 nucleotides. In some embodiments, the length of the scaffold is 70, 71, 72, 73, 74, 75, 76, 77, 78, 79, or 80 nucleotides.

In some embodiments, the scaffold domain may also comprise domain ‘b1*’, which is complementary to domain ‘b1’. Domain ‘b1*’ may be 5 (e.g., contiguous) nucleotides (+/−10 nt). In some embodiments, the length of domain ‘b1*’ is 1-100 nucleotides is 1-100 nucleotides, or 5-100 nucleotides (e.g., 1-10, 1-20, 1-30, 1-40, 1-50, 1-60, 1-70, 1-80, 1-90, 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘b1*’ is 1, 2, 3, 4, 5, 6, 7, 8, 9, 10, 11, 12, 13, 14, 15, 16, 17, 18, 19, or 20 nucleotides.

### Trigger Nucleic Acid

The total length of the trigger nucleic acid, the length of each domain of the trigger nucleic acid, and the orientation of each domain of the trigger nucleic acid depends on the particular configuration of the switch-gRNA.

For switch-gRNAs in which the guide domain ‘a’ is fully sequestered, the position of domain ‘x’ relative to domain ‘a*’ may determine the configuration of the trigger nucleic acid. For example, if domain ‘x’ is located at the 3′ end of the switch-gRNA, downstream of domain ‘a*’ (as shown, for example, in FIGS. 7A and 7C), then domain ‘x*’ of the trigger nucleic acid may be located 5′ from (upstream from) domain ‘a’ of the trigger nucleic acid. As another example, if domain ‘x’ is located at the 5′ end of the switch-gRNA, upstream from domain ‘a*’ (as shown, for example, in FIGS. 8A and 8C), then domain ‘x*’ of the trigger nucleic acid may be located 3′ from (downstream from) domain ‘a’ of the trigger nucleic acid. As yet another example, if domain ‘y’ of the switch-gRNA is located upstream from domain ‘a*’ (as shown, for example, in FIGS. 7B and 7D), then domain ‘y*’ of the trigger nucleic acid may be located 3′ from (downstream from) domain ‘a’ of the trigger nucleic acid. As yet another example, if domain ‘y’ of the switch-gRNA is located downstream from domain ‘a*’ (as shown, for example, in FIGS. 8B and 8D), then domain ‘y*’ of the trigger nucleic acid may be located 5′ from (upstream from) domain ‘a’ of the trigger nucleic acid. As shown in FIGS. 7C-7D and 8C-8D, the trigger nucleic acid may also comprise domain ‘b1’ which is complementary to domain ‘b1*.’ In some instances, domain ‘b1’ is located 3′ from (downstream from) domain ‘a’ on the trigger nucleic acid, while domain ‘b1*’ is located 5′ from (upstream from) domain ‘a*’ of the switch-gRNA.

For the switch-gRNA configurations in which the guide domain ‘a’ is not sequestered but instead is blocked by an adjacent duplex that includes domain ‘z*’ (see, e.g., FIG. 9A), the position of domain ‘z*’ relative to domain ‘x’ and/or domain ‘y’ may determine the configuration of the trigger nucleic acid. For example, if domain ‘x’ is located at the 3′ end of the switch-gRNA, downstream of domain ‘z*’ (as shown, for example, in FIG. 9A), then domain ‘x*’ of the trigger nucleic acid may be located 5′ from (upstream from) domain ‘z’ of the trigger nucleic acid. As another example, if domain ‘x’ is located at the 5′ end of the switch-gRNA, upstream of domain ‘z*’ (as shown, for example, in FIG. 10A), then domain ‘x*’ of the trigger nucleic acid may be located 3′ from (downstream from) domain ‘z’ of the trigger nucleic acid. As yet another example, if domain ‘y’ of the switch-gRNA is located upstream from domain ‘z*’ (as shown, for example, in FIG. 9B), then domain ‘y*’ of the trigger nucleic acid may be located 3′ from (downstream from) domain ‘z’ of the trigger nucleic acid. As yet another example, if domain ‘y’ of the switch-gRNA is located downstream from domain ‘z’″ (as shown, for example, in FIG. 10B), then domain ‘y’″ of the trigger nucleic acid may be located 5′ from (upstream from) domain ‘z’ of the trigger nucleic acid.

For the switch-gRNA configurations in which the guide domain ‘a’ is partially sequestered (see, e.g., FIG. 11A), the position of domain ‘z*’ relative to domain ‘x’, domain ‘a*’ and/or domain ‘y’ may determine the configuration of the trigger nucleic acid. For example, if domain ‘x’ is located at the 3′ end of the switch-gRNA, downstream from domain ‘z*’, and domain ‘z*’ is located downstream from domain ‘a*’ (as shown, for example, in FIG. 11A), then domain ‘x*’ of the trigger nucleic acid may be located 5′ from (upstream from) domain ‘z’ of the trigger nucleic acid, and domain ‘z’ may be located upstream from domain ‘a’ of the trigger nucleic acid. As another example, if domain ‘x’ is located at the 5′ end of the switch-gRNA, upstream of domain ‘a*’, and domain ‘a*’ is located upstream of domain ‘z*’ (as shown, for example, in FIG. 11A), then domain ‘x*’ of the trigger nucleic acid may be located 3′ from (downstream from) domain ‘a’ of the trigger nucleic acid, and domain ‘a’ may be located downstream from domain ‘z’ of the trigger nucleic acid. As yet another example, if domain ‘y’ of the switch-gRNA is located upstream from domain ‘a*’, and domain ‘a*’ is located upstream from domain ‘z*’ (as shown, for example, in FIG. 11B), then domain ‘y*’ of the trigger nucleic acid may be located 3′ from (downstream from) domain ‘a’ of the trigger nucleic acid, and domain ‘a’ may be located downstream from domain ‘z’ of the trigger nucleic acid. As yet another example, if domain ‘y’ of the switch-gRNA is located downstream from domain ‘z*’, and domain ‘z*’ is located downstream from domain ‘a*’ (as shown, for example, in FIG. 12B), then domain ‘y*’ of the trigger nucleic acid may be located 5′ from (upstream from) domain ‘z’ of the trigger nucleic acid, and domain ‘z’ may be located upstream from domain ‘a’ of the trigger nucleic acid.

The length of domain ‘x*’ of the trigger nucleic acid (complementary to domain x of the switch-gRNA) may be 10 (e.g., contiguous) nucleotides (+/−10 nt). In some embodiments, the length of domain ‘x*’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘x*’ is 5, 6, 7, 8, 9, 10, 11, 12, 13, 14, 15, 16, 17, 18, 19, or 20 nucleotides. Domain ‘x*’ may be located at the 5′ end of the trigger nucleic acid or it may be located at the 3′ end of the trigger nucleic acid, depending, in part, on where the complementary domain ‘x’ is located in the switch-gRNA.

Likewise, the length of domain ‘a’ of the trigger nucleic acid (complementary to guide domain ‘a*’ of the switch-gRNA) may be 10 (e.g., contiguous) nucleotides (+/−10 nt). In some embodiments, the length of domain ‘a’ is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘a’ is 10, 11, 12, 13, 14, 15, 16, 17, 18, 19, 20, 21, 22, 23, 24, 25, 26, 27, 28, 29, or 30 nucleotides.

In some embodiments, the length of domain ‘y*’ of the trigger nucleic acid (complementary to the linker domain ‘y’ of the switch-gRNA) is 5 (e.g., contiguous) nucleotides (+/−10 nt). In some embodiments, the length of domain ‘y*’ (or ‘w*’) is 5-100 nucleotides (e.g., 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘y*’ (or ‘w*’) is 5, 6, 7, 8, 9, 10, 11, 12, 13, 14, 15, 16, 17, 18, 19, or 20 nucleotides.

In some embodiments, the length of domain ‘b1’ of the trigger nucleic acid (complementary to domain ‘b1*’ of the switch-gRNA) is 5 (e.g., contiguous) nucleotides (+/−10 nt). In some embodiments, the length of domain ‘b1’ is 1-100 nucleotides is 1-100 nucleotides, or 5-100 nucleotides (e.g., 1-10, 1-20, 1-30, 1-40, 1-50, 1-60, 1-70, 1-80, 1-90, 5-10, 5-20, 5-30, 5-40, 5-50, 5-60, 5-70, 5-80, 5-90, 10-20, 10-30, 10-40, 10-50, 10-60, 10-70, 10-80, 10-90, 10-100, 20-30, 20-40, 20-50, 20-60, 20-70, 20-80, 20-90 or 20-100 nucleotides). In some embodiments, the length of domain ‘b1’ is 1, 2, 3, 4, 5, 6, 7, 8, 9, 10, 11, 12, 13, 14, 15, 16, 17, 18, 19, or 20 nucleotides. Domain ‘b1’ may be located at the 3′ end of the trigger nucleic acid or it may be located elsewhere on the trigger nucleic acid, 3′ and adjacent to domain ‘a’. In some embodiments, domain ‘b1’ may be absent.

Implementation of Switch-gRNA Designs in Domain ‘b,’ ‘c’ and ‘d’

The embodiments provided herein, for example, in FIGS. 7A-12B depict the implementation of the switch in domain ‘a’ for illustration and ease of understanding. It should be understood that any of domains ‘b,’ ‘c’ and ‘d’ (see, e.g., FIG. 1) can be engineered in a similar way (see, e.g., FIGS. 13A and 13B). For the two-component guide RNA, the switch can be implemented in domain ‘b’, ‘c’ and ‘d’ (FIG. 13A). For the single-guide RNA, the switch can be implemented in domain ‘b’ (FIG. 13B).

In addition, multiple switches can be used in combination by incorporating them in different domains of the same gRNA to increase the level of activation control. For example, a switch may be added in domain ‘a’ and ‘b,’ each responding to different triggers to create an AND gate where the guide RNA is activated only if trigger A and B are present.

### Csy4-Mediated Release

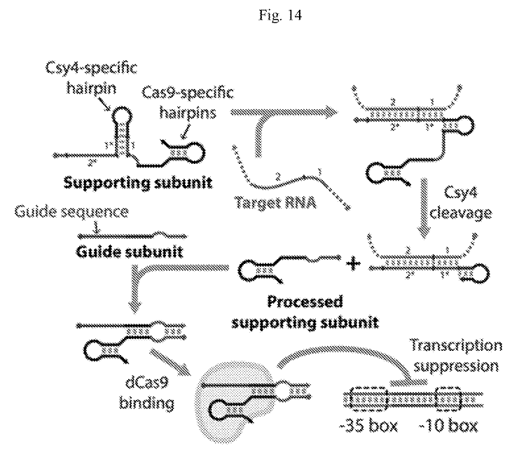

Another embodiment of the present disclosure is depicted in FIG. 14. An RNA endonuclease (Csy4/Cas6) was used to process the engineered inactive tracrRNA of Cas9. Csy4 recognizes RNA molecules with a signature hairpin sequence and cleaves these RNAs at a defined site, the junction of a 15-nt hairpin and a downstream single-stranded region.

In the absence of the input RNA, the tracrRNA folds into a hairpin structure that sequesters the domain necessary to interact with the crRNA (the ˜10-nt domain 1). At the top of this hairpin is the 15-nt hairpin (black) recognized by Csy4. However, since domain 1 downstream of the black hairpin is paired with domain 1*, the tracrRNA is not cleaved by Csy4.

When the input RNA (the trigger) is present, it binds to the inactive tracrRNA and opens the 1:1* portion of the hairpin, making the tracrRNA cleavable by Csy4. The processed tracrRNA is the active form of tracrRNA and therefore can bind the crRNA and form a functional gRNA that can engage Cas9.

It is important to note that the tracrRNA and the crRNA must contain the complementary interacting domain (domain 1) to form a functional gRNA, thus multiple guide/supporting subunit pairs can function orthogonally in one cell.

### Catalytic Circuit

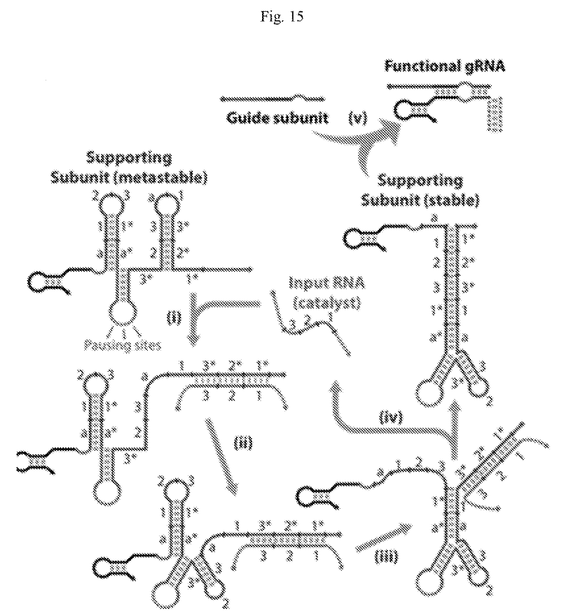

Another embodiment of the present disclosure is depicted in FIG. 15. Co-transcriptional kinetic traps may be engineered in the supporting subunit described above so that its nascent transcript is in a meta-stable state with 3 hairpins (see, e.g., FIG. 15). The left hairpin contains domain ‘a’ that, when exposed, can interact with the guide subunit to form the functional gRNA.

The input RNA can bind domain ‘1*’, 5′ to the right hairpin and open it via strand displacement (FIG. 15, step i). Stem (3-3*) then forms (FIG. 15, step ii) to prime the opening of the left hairpin (FIG. 15, step iii). Further rearrangement displaces the input RNA (FIG. 15, step iv). Overall, the input RNA catalyzes the refolding of the supporting subunit from the metastable state to the stable state. The refolded supporting subunit, with exposed domain ‘a,’ can then hybridize with the guide subunit to form the gRNA (FIG. 15, step v).

### Conditional Activation of DNA-Guided Nucleases Using NgAgo

Unlike RNA-guided endonucleases, the Argonaute protein from Natronobacterium gregoryi (NgAgo) is a DNA-guided endonuclease. NgAgo binds 5′ phosphorylated ssDNA of ˜24 nucleotides guide (gDNA) to recognize and cleave complementary DNA strands. Provided herein are engineered gDNAs that can be activated by specific nucleic acid sequences. This allows controlling the activity of DNA guided endonucleases, including their mutated forms.

In some embodiments, a gDNA has a length of 10 to 100 nucleotides. For example, a gDNA may have a length of 10-90, 10-80, 10-70, 10-60, 10-50, 10-40, 10-35, 10-30, 10-25, 10-20, 10-15, 15-100, 15-90, 15-80, 15-70, 15-60, 15-50, 15-40, 15-35, 15-30, 15-25, 15-20, 20-100, 20-90, 20-80, 20-70, 20-60, 20-50, 20-40, 20-35, 20-30 or 20-25 nucleotides. In some embodiments, a gDNA has a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Longer gRNAs are encompassed by the present disclosure.

DNA complexes were engineered to sequester guide DNAs and release them only if specific RNA/DNA molecules are present. When guide DNAs are sequestered, the nuclease NgAgo is in the apo state and therefore inactive. The release of the gDNA is triggered by a mechanism referred to as toehold-mediated strand displacement. When a specific single-stranded endogenous or synthetic DNA or RNA sequence is present (referred to as the trigger), the trigger binds to the engineered DNA complex and induces a strand displacement reaction that releases the single-stranded guide DNA. Once the gDNA is released, it binds to NgAgo and activate the nuclease. The engineered DNA complexes can be designed to implement logic operations such as AND OR gates.

DNA complexes may be produced in vitro and subsequently introduced into target cells or used in vitro.

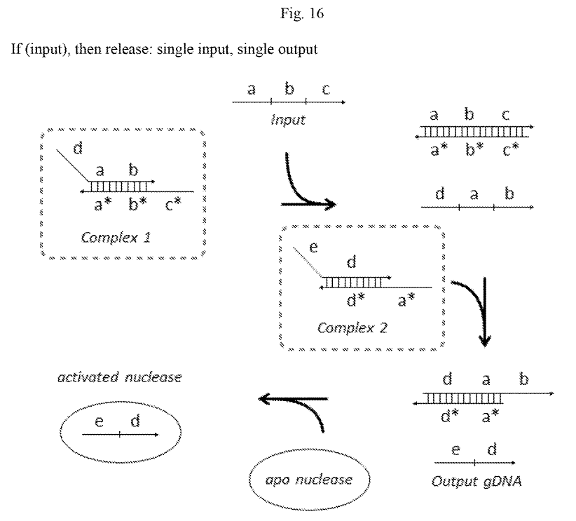

As shown in FIG. 16, when the input sequence a-b-c (the trigger) is present, it binds to complex 1 and displaces the intermediate DNA strand d-a-b, which in turn binds to complex 2 and displaces the output gDNA e-d. When the gDNA is released, it binds to NgAgo and activate the nuclease. The intermediate strand d-a-b ensures that the sequence of the trigger a-b-c and of the gDNA e-d are unrelated. The approximate length of the gDNA e-d is 25 nucleotides (+/−10 nt). Thus, in some embodiments, the length of the gDNA is e-d 15-35 nucleotides (e.g., 15, 20, 25, 30 or 35 nucleotides). In some embodiments, the length of the gDNA e-d is 24 nucleotides.

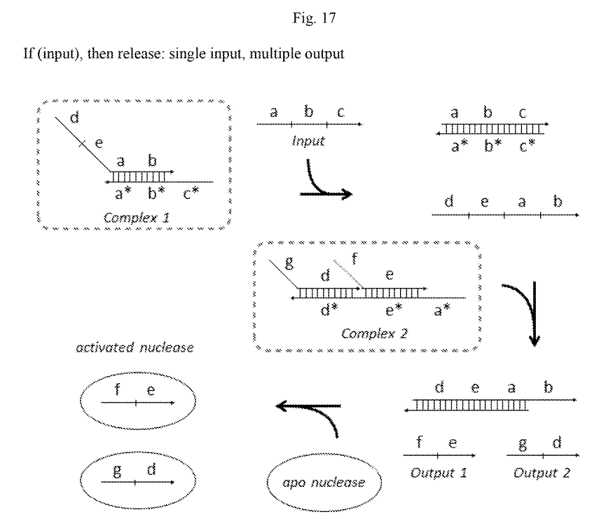

As shown in FIG. 17, when the input sequence a-b-c (the trigger) is present, it binds to complex 1 and displaces the intermediate DNA strand d-e-a-b, which in turn binds to complex 2 and displaces the output gDNAf-e (output 1) and g-d (output 2). When the gDNAs are released, they binds to NgAgo proteins and activate them. The intermediate strand d-e-a-b ensures that the sequence of the trigger a-b-c and of the gDNAs are unrelated. The approximate length of each of the gDNAf-e and g-d is 25 nucleotides (+/−10 nt). Thus, in some embodiments, the length of each of the gDNAf-e and g-d is 15-35 nucleotides (e.g., 15, 20, 25, 30 or 35 nucleotides). In some embodiments, the length of each of the gDNAf-e and g-d is 24 nucleotides.

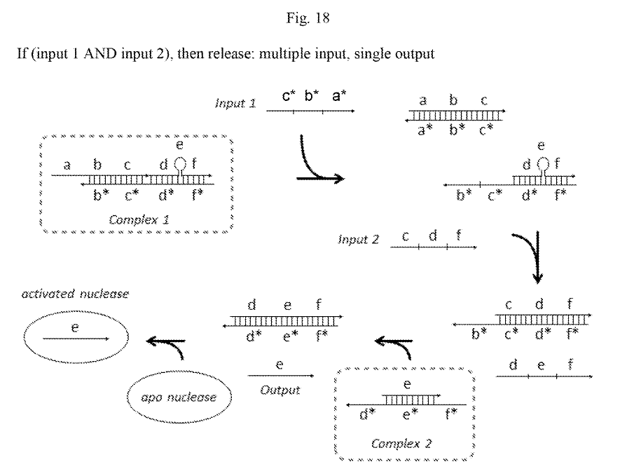

As shown in FIG. 18, when input sequence 1 a*-b*-c* is present, it binds to complex 1 and displaces the DNA strand a-b-c, which in turn enables the binding of input sequence 2 c-d-f to complex 1 because sequence c* is now accessible. Then, the binding of input sequence 2 displaces the intermediate DNA strand d-e-f which in turn binds to complex 2 and displaces the guide DNA e. When the gDNA is released, it binds to NgAgo and activates the nuclease. The approximate length of the gDNA e is 25 nucleotides (+/−10 nt). Thus, in some embodiments, the length of the gDNA e is 15-35 nucleotides (e.g., 15, 20, 25, 30 or 35 nucleotides). In some embodiments, the length of the gDNA e is 24 nucleotides.

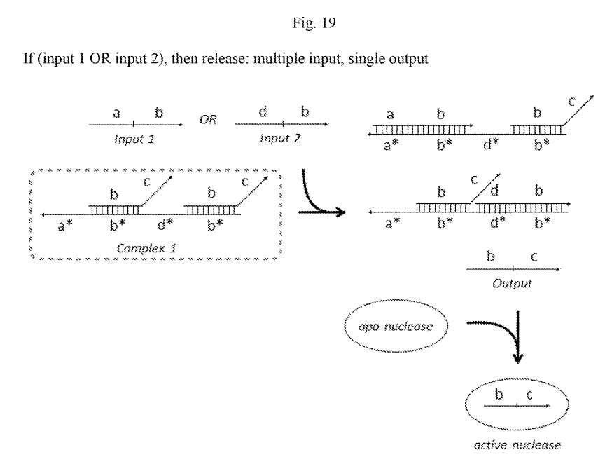

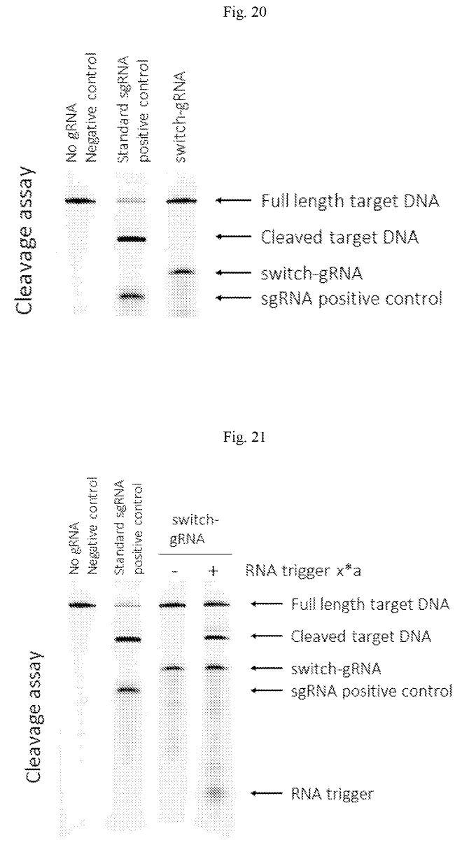

As shown in FIG. 19, when input sequence 1 a-b is present, it binds to complex 1 and displaces the gDNA strand b-c. Similarly, when sequence 2 d-b is present, it binds to complex 1 and displaces the gDNA strand b-c. When gDNA are released, they bind to NgAgo and activate the nuclease. The approximate length of the gDNA b-c is 25 nucleotides (+/−10 nt). Thus, in some embodiments, the length of the gDNA b-c is 15-35 nucleotides (e.g., 15, 20, 25, 30 or 35 nucleotides). In some embodiments, the length of the gDNA b-c is 24 nucleotides.

### Nucleic Acids

An “engineered nucleic acid” is a nucleic acid (e.g., at least two nucleotides covalently linked together, and in some instances, containing phosphodiester bonds, referred to as a phosphodiester “backbone”) that does not occur in nature. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with (also referred to as “binding to,” e.g., transiently or stably) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid includes a murine nucleotide sequence, a bacterial nucleotide sequence, a human nucleotide sequence, a viral nucleotide sequence, or a combination of any two or more of the foregoing sequences.

A “domain” refers to a discrete, contiguous sequence of nucleotides or nucleotide base pairs, depending on whether the domain is unpaired (contiguous stretch of nucleotides that are not bound to complementary nucleotides) or paired (contiguous stretch of nucleotide base pairs—nucleotides bound to complementary nucleotides), respectively. In some embodiments, a domain is described as having multiple subdomains for the purpose of defining intramolecular (within the same molecular species) and intermolecular (between two separate molecular species) complementarity. One domain (or one subdomain) is “complementary to” another domain (or another subdomain) if one domain contains nucleotides that base pair (hybridize/bind through Watson-Crick nucleotide base pairing) with nucleotides of the other domain such that the two domains form a paired (double-stranded) or partially-paired molecular species/structure. Complementary domains need not be perfectly (100%) complementary to form a paired structure, although perfect complementarity is provided, in some embodiments. Thus, a primer that is “complementary” to a particular domain binds to that domain, for example, for a time sufficient to initiate polymerization in the presence of polymerase.

In some embodiments, an engineered nucleic acid of the present disclosure may comprise a backbone other than a phosphodiester backbone. For example, an engineered nucleic acid, in some embodiments, may comprise phosphoramide, phosphorothioate, phosphorodithioate, O-methylphophoroamidite linkages, peptide nucleic acids or a combination of any two or more of the foregoing linkages. An engineered nucleic acid may be single-stranded (ss) or double-stranded (ds), as specified, or an engineered nucleic acid may contain portions of both single-stranded and double-stranded sequence. In some embodiments, an engineered nucleic acid contains portions of triple-stranded sequence. An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids are known in the art and may be used in accordance with the present disclosure.

Domains or other discrete nucleotide sequences are considered “adjacent” to each other if they are contiguous with each other (there are no nucleotides separating the two domains), or if they are within 50 nucleotides (e.g., 1-50, 1-40, 1-30, 1-20, 1-10, 1-5) of each other. That is, in some embodiments, two domains may be considered adjacent if the two domains are separated from each other by 1, 2, 3, 4, 5, 6, 7, 8, 9, 10, 11, 12, 13, 14, 15, 16, 17, 18, 19, 20, 25, 30, 35, 40, 45 or 50 nucleotides.

Nucleotide domains and subdomains are described in terms of a 3′ and/or 5′ position relative to one another, or relative to the entire length of a nucleic acid. For example, with reference to FIG. 5A, as an example, the inactive gRNA includes a 5′ paired domain and a 3′ unpaired domain. The 3′ unpaired domain is labeled ‘x’, and the 5′ paired domain includes the terminal loop structure and is labeled with a ‘1’ subdomain bound to a ‘1*’ subdomain. As another example, the single-stranded trigger depicted in FIG. 5A includes a 5′ unpaired domain ‘x*’ contiguous with a 3′ unpaired domain ‘1,’ where domain ‘x*’ is complementary to domain ‘x’ and domain ‘1’ is complementary to domain ‘1*’.

An inactive gRNA of the present disclosure typically include at least one hairpin structure, which is a stretch of contiguous nucleotides that folds through intramolecular base pairing to form a paired domain flanked by a unpaired linear domain and an unpaired loop domain, as shown, for example, in FIG. 5A (Inactive gRNA).

An “unpaired domain” of a nucleic acid (e.g., gRNA) refers to a sequence of nucleotides that is not bound to a complementary sequence of nucleotides. Single-stranded nucleic acids, for example, are considered “unpaired” nucleic acids.

A “paired domain” of a nucleic acid (e.g., gRNA) refers to a sequence of nucleotides bound to a complementary sequence of nucleotides (e.g., Watson-Crick nucleobase pairing). Double-stranded nucleic acids, for example, are considered “paired” nucleic acids.

A “loop domain” of nucleic acid (e.g., gRNA) refers to an unpaired domain that form a loop-like structure at the end (adjacent to) a 5′ paired domain. That is, a loop domain links complementary domains of a nucleic acid to form a 5′ paired domain. Thus, in some embodiments, a “loop domain” may be referred to as a “linker domain.” A loop domain may be substituted with a linker domain (e.g., 1-5 nucleotides in length).

### Cells

Nucleic acids of the present disclosure may be introduced into a variety of different cells, in vivo or in vitro. Examples of cells into which nucleic acids may be introduced include, but are not limited to, mammalian cells, insect cells, bacterial cells (e.g., Escherichia coli cells) and yeast cells (e.g., Saccharomyces cerevisiae cells). Mammalian cells may be human cells, primate cells (e.g., vero cells), rat cells (e.g., GH3 cells, OC23 cells) or mouse cells (e.g., MC3T3 cells), for example. There are a variety of human cell lines, including, but are not limited to, HEK cells (e.g., HEK 293 or HEK 293T cells), HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells.

In some embodiments, nucleic acids are introduced in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, 2006 Cell 126 (4): 663-76, incorporated by reference herein). Human induced pluripotent stem cell express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature. In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell.

An nucleic acid may be introduced into a cell by methods, such as, for example, electroporation (see, e.g., Heiser W. C. Transcription Factor Protocols: Methods in Molecular Biology™ 2000; 130: 117-134), chemical (e.g., calcium phosphate or lipid), transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), or microinjection of purified DNA directly into the nucleus of the cell (see, e.g., Capecchi M. R. Cell. 1980 November; 22(2 Pt 2): 479-88).

Mammalian cells (e.g., human cells) modified to comprise nucleic acids of the present disclosure may be cultured (e.g., maintained in cell culture) using conventional mammalian cell culture methods (see, e.g., Phelan M. C. Curr Protoc Cell Biol. 2007 September; Chapter 1: Unit 1.1, incorporated by reference herein). For example, cells may be grown and maintained at an appropriate temperature and gas mixture (e.g., 37° C., 5% CO₂ for mammalian cells) in a cell incubator. Culture conditions may vary for each cell type. For example, cell growth media may vary in pH, glucose concentration, growth factors, and the presence of other nutrients. Growth factors used to supplement media are often derived from the serum of animal blood, such as fetal bovine serum (FBS), bovine calf serum, equine serum and/or porcine serum. In some embodiments, culture media used as provided herein may be commercially available and/or well-described (see, e.g., Birch J. R., R. G. Spier (Ed.) Encyclopedia of Cell Technology, Wiley. 411-424, 2000; Keen M. J. Cytotechnology 17: 125-132, 1995; Zang, et al. Bio/Technology. 13: 389-392, 1995). In some embodiments, chemically defined media is used.

### Compositions

It should be understood that in each of the following embodiments, the different domains (containing a sequence of nucleotides) that make up a particular nucleic acid strand are denoted by a letter-domain a, domain b, domain b1, domain w, domain x, domain y, or domain z. A domain letter accompanied by a “*” denotes complementarity. Thus, domain a* is complementary to domain a (domain a* comprises a nucleotide sequence that is complementary to, and thus capable of binding/hybridizing to, domain a). The length and/or nucleotide composition of each domain may vary, as indicated below. A scaffold domain, in some embodiments, comprises a nucleotide sequence used for Cas9-binding. In some embodiments, the scaffold domain comprises the following sequence: 5′ GUUUUAGAGCUAGAAAUAGCAAGUUAAAAUAAGGCUAGUCCGUUAUCAACU UGAAAAAGUGGCACCGAGUCGGUGC 3′ (SEQ ID NO: 37).

In some embodiments, the present disclosure provides a composition comprising:

(a) switch guide RNA (gRNA) strand comprising, in the 5′ to 3′ direction, guide domain a, scaffold domain b, unpaired domain w, domain a*, and unpaired toehold domain x,

wherein guide domain a and domain a* are complementary to each other; and

(b) a trigger strand comprising, in the 5′ to 3′ direction, domain x* and domain a,

wherein domain x* and domain a are respectively complementary to domain x and domain a* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 7A.

In other embodiments, the present disclosure provides a composition comprising:

(a) switch guide RNA (gRNA) strand comprising, in the 5′ to 3′ direction, guide domain a, scaffold domain b, unpaired domain y, and domain a*,

wherein guide domain a and domain a* are complementary to each other; and

(b) a trigger strand comprising, in the 5′ to 3′ direction, domain a and domain y*,

wherein domain a and domain y* are respectively complementary to domain a* and domain y of the switch gRNA strand. In some embodiments, domain y has a length of at least 5 nucleotides (e.g., 5, 10, 15, 20, at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). See, e.g., FIG. 7B.

In yet other embodiments, the present disclosure provides a composition comprising:

(a) switch guide RNA (gRNA) strand comprising, in the 5′ to 3′ direction, guide domain a, scaffold domain b1, scaffold domain b, unpaired domain w, domain b1*, domain a*, and unpaired toehold domain x,

wherein guide domain a and domain a* are complementary to each other, and scaffold domain b1 and domain b1* are complementary to each other; and

(b) a trigger strand comprising, in the 5′ to 3′ direction, domain x*, domain a, and domain b1,

wherein domain x*, domain a and domain b1 are respectively complementary to domain x, domain a*, and domain b1* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain b1* has a length of at least 1 nucleotide (e.g., at least 2, 3, 4, 5, 6, 7. 8, 9, 10, 1-5, or 1-10 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 7C.

In still other embodiments, the present disclosure provides a composition comprising:

(a) switch guide RNA (gRNA) strand comprising, in the 5′ to 3′ direction, guide domain a, scaffold domain b1, scaffold domain b, unpaired domain y, domain b1*, and domain a*,

wherein guide domain a and domain a* are complementary to each other, and scaffold domain b1 and domain b1* are complementary to each other; and

(b) a trigger strand comprising, in the 5′ to 3′ direction, domain a, domain b1, and domain y*,

wherein domain a, domain b1, and domain y* are respectively complementary to domain a*, domain b1*, and domain y of the switch gRNA strand. In some embodiments, domain y has a length of at least 5 nucleotides (e.g., 5, 10, 15, 20, at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain b1* has a length of at least 1 nucleotide (e.g., at least 2, 3, 4, 5, 6, 7. 8, 9, 10, 1-5, or 1-10 nucleotides). See, e.g., FIG. 7D.

In some embodiments, the present disclosure provides a composition comprising: (a) switch guide RNA (gRNA) strand comprising, in the 5′ to 3′ direction, unpaired toehold domain x, domain a*, unpaired domain w, guide domain a, and scaffold domain b,

wherein guide domain a and domain a* are complementary to each other; and

(b) a trigger strand comprising, in the 5′ to 3′ direction, domain a and domain x*,

wherein domain a and domain x* are respectively complementary to domain a* and domain x of the switch gRNA strand. In some embodiments, domain w has a length of at least 4 nucleotides (e.g., 4, 5, 10, 15, 20, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 8A.

In other embodiments, the present disclosure provides a composition comprising:

(a) switch guide RNA (gRNA) strand comprising, in the 5′ to 3′ direction, domain a*, unpaired domain y, unpaired domain w, guide domain a, and scaffold domain b,

wherein guide domain a and domain a* are complementary to each other; and

(b) a trigger strand comprising, in the 5′ to 3′ direction, domain y* and domain a,

wherein domain y* and domain a are respectively complementary to domain y and domain a* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domain y has a length of at least 5 nucleotides (e.g., 5, 10, 15, 20, at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). See, e.g., FIG. 8B.

In yet other embodiments, the present disclosure provides a composition comprising:

(a) switch guide RNA (gRNA) strand comprising, in the 5′ to 3′ direction, unpaired toehold domain x, domain b1*, domain a*, unpaired domain w, guide domain a, scaffold domain b1, and scaffold domain b,

wherein guide domain a and domain a* are complementary to each other, and scaffold domain b1 and domain b1* are complementary to each other; and

(b) a trigger strand comprising, in the 5′ to 3′ direction, domain a, domain b1, and domain x*,

wherein domain a, domain b1, and domain x* are respectively complementary to domain a*, domain b1*, and domain x of the switch gRNA strand. In some embodiments, domain w has a length of at least 4 nucleotides (e.g., 4, 5, 10, 15, 20, at least 5, at least 10, 1-20, or 1-50 nucleotides). In some embodiments, domain x has a length of at least 5 nucleotides (e.g., 5, 10, 15, 20, at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain b1* has a length of at least 1 nucleotide (e.g., at least 2, 3, 4, 5, 6, 7. 8, 9, 10, 1-5, or 1-10 nucleotides). See, e.g., FIG. 8C.

In still other embodiments, the present disclosure provides a composition comprising:

(a) switch guide RNA (gRNA) strand comprising, in the 5′ to 3′ direction, domain b1*, domain a*, unpaired domain y, unpaired domain w, guide domain a, scaffold domain b1, and scaffold domain b,

wherein guide domain a and domain a* are complementary to each other, and scaffold domain b1 and domain b1* are complementary to each other; and

(b) a trigger strand comprising, in the 5′ to 3′ direction, domain y*, domain a, and domain b1,

wherein domain y*, domain a, and domain b1 are respectively complementary to domain y, domain a*, and domain b1* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domain y has a length of at least 5 nucleotides (e.g., 5, 10, 15, 20, at least 2, at least 5, at least 10, 1-20, or 1-50 nucleotides). In some embodiments, domain a* has a length of 1-20 nucleotides (e.g., 1, 2, 3, 4, 5, 6, 7, 8, 9, 10, 11, 12, 13, 14, 15, 16, 17, 18, 19 or 20 nucleotides). In some embodiments, domain b1* has a length of at least 1 nucleotide (e.g., at least 2, 3, 4, 5, 6, 7. 8, 9, 10, 1-5, or 1-10 nucleotides). See, e.g., FIG. 8D.

In some embodiments, the present disclosure provides a composition comprising:

(a) switch guide RNA (gRNA) strand comprising, in the 5′ to 3′ direction, domain z, guide domain a, scaffold domain b, unpaired domain w, domain z*, and unpaired toehold domain x,

wherein domain z and domain z* are complementary to each other; and

(b) a trigger strand comprising, in the 5′ to 3′ direction, domain x* and domain z,

wherein domain x* and domain z are respectively complementary to domain x and domain z* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domains z and z* have a length of at least 5 nucleotides (e.g., 5, 10, 15, 20, at least 2, at least 5, at least 10, 1-20, or 1-50 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 9A.

In other embodiments, the present disclosure provides a composition comprising:

(a) switch guide RNA (gRNA) strand comprising, in the 5′ to 3′ direction, domain z, guide domain a, scaffold domain b, unpaired domain w, and domain z*,

wherein domain z and domain z* are complementary to each other; and

(b) a trigger strand comprising, in the 5′ to 3′ direction, domain z and domain y*,

wherein domain z and domain y* are respectively complementary to domain z* and domain y of the switch gRNA strand. In some embodiments, domains z and z* have a length of at least 5 nucleotides (e.g., 5, 10, 15, 20, at least 2, at least 5, at least 10, 1-20, or 1-50 nucleotides). In some embodiments, domain y has a length of at least 5 nucleotides (e.g., 5, 10, 15, 20, at least 2, at least 5, at least 10, 1-20, or 1-50 nucleotides). See, e.g., FIG. 9B.

In yet other embodiments, the present disclosure provides a composition comprising:

(a) switch guide RNA (gRNA) strand comprising, in the 5′ to 3′ direction, domain x, guide domain z*, domain w, domain z, guide domain a, and scaffold domain b,

wherein domain z and domain z* are complementary to each other; and

(b) a trigger strand comprising, in the 5′ to 3′ direction, domain z and domain x*,

wherein domain z and domain x* are respectively complementary to domain z* and domain x of the switch gRNA strand. In some embodiments, domain w has a length of at least 4 nucleotides (e.g., 4, 5, 10, 15, 20, at least 5, at least 10, 1-20, or 1-50 nucleotides). In some embodiments, domain x has a length of at least 5 nucleotides (e.g., 5, 10, 15, 20, at least 2, at least 5, at least 10, 1-20, or 1-50 nucleotides). In some embodiments, domain z* has a length of at least 5 nucleotides (e.g., 5, 10, 15, 20, at least 2, at least 5, at least 10, 1-20, or 1-50 nucleotides). See, e.g., FIG. 10A.

In still other embodiments, the present disclosure provides a composition comprising:

(a) switch guide RNA (gRNA) strand comprising, in the 5′ to 3′ direction, domain z*, guide domain y, domain z, guide domain a, and scaffold domain b,

wherein domain z and domain z* are complementary to each other; and

(b) a trigger strand comprising, in the 5′ to 3′ direction, domain y* and domain z,

wherein domain y* and domain z are respectively complementary to domain y and domain z* of the switch gRNA strand. In some embodiments, domain y has a length of at least 5 nucleotides (e.g., 5, 10, 15, 20, at least 2, at least 5, at least 10, 1-20, or 1-50 nucleotides). In some embodiments, domain z* has a length of at least 5 nucleotides (e.g., 5, 10, 15, 20, at least 2, at least 5, at least 10, 1-20, or 1-50 nucleotides). See, e.g., FIG. 10B.

In some embodiments, the present disclosure provides a composition comprising:

(a) switch guide RNA (gRNA) strand comprising, in the 5′ to 3′ direction, domain z, guide domain a, scaffold domain b, unpaired domain w, domain a*, domain z*, and unpaired toehold domain x,

wherein domain z and domain z* are complementary to each other, and wherein guide domain a is only partially complementary to domain a*; and

(b) a trigger strand comprising, in the 5′ to 3′ direction, domain x*, domain z, and domain a,

wherein domain x*, domain z, and domain a are respectively complementary to domain x, domain z*, and domain a* of the switch gRNA strand. In some embodiments, the switch gRNA strand does not include domain w. In some embodiments, domain w has a length of at least 1 nucleotide (e.g., at least 2, at least 5, at least 10, 1-20, or 1-50 nucleotides). In some embodiments, domain a*+domain z* has a length of at least 5 nucleotides (e.g., 5, 10, 15, 20, at least 2, at least 5, at least 10, 1-20, or 1-50 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 11A.

In other embodiments, the present disclosure provides a composition comprising:

(a) switch guide RNA (gRNA) strand comprising, in the 5′ to 3′ direction, domain z, guide domain a, scaffold domain b, unpaired domain y, domain a*, and domain z*,

wherein domain z and domain z* are complementary to each other, and wherein guide domain a is only partially complementary to domain a*; and

(b) a trigger strand comprising, in the 5′ to 3′ direction, domain z, domain a, and domain y*,

wherein domain z, domain a, and domain y*are respectively complementary to domain z*, domain a*, and domain y of the switch gRNA strand. In some embodiments, domain a*+domain z* has a length of at least 5 nucleotides (e.g., 5, 10, 15, 20, at least 2, at least 5, at least 10, 1-20, or 1-50 nucleotides). In some embodiments, domain y has a length of at least 5 nucleotides (e.g., 5, 10, 15, 20, at least 2, at least 5, at least 10, 1-20, or 1-50 nucleotides). See, e.g., FIG. 11B.

In yet other embodiments, the present disclosure provides a composition comprising:

(a) switch guide RNA (gRNA) strand comprising, in the 5′ to 3′ direction, unpaired toehold domain x, domain a*, domain z*, unpaired domain w, domain z, guide domain a, and scaffold domain b,

wherein domain z and domain z* are complementary to each other, and wherein guide domain a is only partially complementary to domain a*; and

(b) a trigger strand comprising, in the 5′ to 3′ direction, domain z, domain a, and domain x*,

wherein domain z, domain a, and domain x* are respectively complementary to domain z*, domain a*, and domain x of the switch gRNA strand. In some embodiments, domain w has a length of at least 4 nucleotide (e.g., 4, 5, 10, 15, 20, at least 5, at least 10, 1-20, or 1-50 nucleotides). In some embodiments, domain a*+domain z* has a length of at least 5 nucleotides (e.g., 5, 10, 15, 20, at least 2, at least 5, at least 10, 1-20, or 1-50 nucleotides). In some embodiments, domain x has a length of at least 5 nucleotides (e.g., 5, 10, 15, 20, at least 2, at least 5, at least 10, 1-20, or 1-50 nucleotides). See, e.g., FIG. 12A.

In still other embodiments, the present disclosure provides a composition comprising:

(a) switch guide RNA (gRNA) strand comprising, in the 5′ to 3′ direction, domain a*, domain z*, unpaired domain w, domain z, guide domain a, and scaffold domain b,

wherein domain z and domain z* are complementary to each other, and wherein guide domain a is only partially complementary to domain a*; and

(b) a trigger strand comprising, in the 5′ to 3′ direction, domain y*, domain z, and domain a,

wherein domain y*, domain z, and domain a are respectively complementary to domain y*, domain z, and domain a of the switch gRNA strand. In some embodiments, domain a*+domain z* has a length of at least 5 nucleotides (e.g., 5, 10, 15, 20, at least 2, at least 5, at least 10, 1-20, or 1-50 nucleotides). In some embodiments, domain y has a length of at least 5 nucleotides (e.g., 5, 10, 15, 20, at least 2, at least 5, at least 10, 1-20, or 1-50 nucleotides). See, e.g., FIG. 12B.

### Applications

The nucleic acids/compositions of the present disclosure may be used in a variety of applications, including, without limitation, synthetic lethality screens, CRISPRi/a (inactivation/activation) screens, CRISPR/Cas9 knockout arrayed screening assays, off-target detection assays, cell line generation (e.g., enrichment for knockin events), pooled-to-arrayed screening and barcoding. Other applications and uses of the nucleic acids include, without limitation, single cell analysis, isolation of specific mutations among a population of cells, analyzing drug resistance in cells, antibody generation, isolation of clones capable of high protein production, and genome-wide tagging and isolation. See, e.g., Mali P, et al. Science. 2013 Feb. 15; 339(6121):823-6; Want T, et al. Science. 2014 Jan. 3; 343(6166):80-4; Shalem O, et al. Science. 2014 Jan. 3; 343(6166):84-7; Koike-Yusa H, et al. Nat Biotechnol. 2014 March; 32(3):267-73; and Zhou Z, et al. Nature. 2014 May 22; 509(7501):487-91.

In some embodiments, nucleic acids/compositions of the present disclosure may be used to generate a knock-out cell or organism.

In some embodiments, nucleic acids/compositions may be used to activate or repress a target gene. One feature of Cas9, for example, is its ability to bind target DNA independently of its ability to cleave target DNA. Specifically, both RuvC- and HNH-nuclease domains can be rendered inactive by point mutations (D10A and H840A in SpCas9), resulting in a nuclease dead Cas9 (dCas9) molecule that cannot cleave target DNA. The dCas9 molecule retains the ability to bind to target DNA based on the gRNA targeting sequence. In some embodiments, dCas9 may be targeted to transcriptional start sites to “repress” or “knock-down” transcription by blocking transcription initiation. In some embodiments, dCas9 may be tagged with transcriptional repressors or activators, and these dCas9 fusion proteins may be targeted to a promoter region, resulting in robust transcription repression or activation of downstream target genes. The simplest dCas9-based activators and repressors include dCas9 fused directly to a single transcriptional activator, A (e.g., VP64) or transcriptional repressors, R (e.g., KRAB).

In some embodiments, nucleic acids/compositions may be used for genome-wide screening applications.

The present disclosure also provides composition and kits comprising at least one of the nucleic acids and/or compositions of the present disclosure.

### Additional Embodiments

The present disclosure further provides embodiments encompassed by the following numbered paragraphs:

1. A composition, comprising:

(a) an inactive guide ribonucleic acid (RNA) comprising a secondary structure that prevents binding of the gRNA to a cognate RNA-guided endonuclease; and optionally

(b) a trigger nucleic acid capable of binding to the inactive gRNA and disrupting the secondary structure to produce an active form of the gRNA that is capable of binding to the cognate RNA-guided endonuclease (see, e.g., FIG. 5A).

2. The composition of paragraph 1 further comprising the RNA-guided endonuclease.

3. The composition of paragraph 1 or 2, wherein the RNA-guided endonuclease is Cas9, Cpf1 or C2c2.

4. A composition, comprising:

(a) an inactive guide ribonucleic acid (RNA) comprising
(i) an unpaired hairpin loop domain,
(ii) a paired stem domain located adjacent to the unpaired hairpin loop domain and comprising a first subdomain complementary to and bound to a second subdomain that, when not bound to the first subdomain, associates with an RNA-guided endonuclease, and
(iii) an unpaired toehold domain contiguous with the first subdomain of the paired stem domain; and optionally

(b) a trigger nucleic acid comprising (i) an unpaired subdomain complementary to the first subdomain of the paired stem domain of the inactive gRNA and (ii) an unpaired subdomain complementary to the toehold domain of the inactive gRNA (see, e.g., FIGS. 7A, 7C, 8A and 8C).

5. A composition, comprising:

(a) an inactive guide ribonucleic acid (RNA) comprising
(i) an unpaired hairpin loop domain, and
(ii) a paired stem domain located adjacent to the unpaired hairpin loop domain and comprising a first subdomain complementary to and bound to a second subdomain that, when not bound to the first subdomain, associates with an RNA-guided endonuclease; and optionally

(b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA (see, e.g., FIGS. 7B, 7D, 8B and 8D).

6. A composition, comprising:

(a) an inactive guide ribonucleic acid (RNA) comprising
(i) an unpaired hairpin loop domain,
(ii) a paired stem domain located adjacent to the unpaired hairpin loop domain and comprising a first subdomain contiguous with a second subdomain, and a third subdomain contiguous with the fourth subdomain, wherein the first subdomain and the second subdomain are respectively complementary to and bound to the third subdomain and the fourth subdomain, and wherein fourth subdomain, when not bound to the second subdomain, associates with an RNA-guided endonuclease, and
(iii) an unpaired toehold domain contiguous with the second subdomain of the paired stem domain; and optionally

(b) a trigger nucleic acid comprising (i) an unpaired subdomain complementary to the first subdomain of the paired stem domain of the inactive gRNA, (ii) an unpaired subdomain complementary to the second subdomain of the paired stem domain of the inactive gRNA, and (iii) an unpaired subdomain complementary to the toehold domain of the inactive gRNA (see, e.g., FIGS. 11A and 12A).

7. A composition, comprising:

(a) an inactive guide ribonucleic acid (RNA) comprising
(i) an unpaired hairpin loop domain, and
(ii) a paired stem domain located adjacent to the unpaired hairpin loop domain and comprising a first subdomain contiguous with a second subdomain, and a third subdomain contiguous with the fourth subdomain, wherein the first subdomain and the second subdomain are respectively complementary to and bound to the third subdomain and the fourth subdomain, and wherein fourth subdomain, when not bound to the second subdomain, associates with an RNA-guided endonuclease; and optionally

(b) a trigger nucleic acid comprising (i) an unpaired subdomain complementary to the hairpin loop domain of the inactive gRNA, (ii) an unpaired subdomain complementary to the first subdomain of the paired stem domain of the inactive gRNA, and (iii) an unpaired subdomain complementary to the second subdomain of the paired stem domain of the inactive gRNA (see, e.g., FIGS. 11B and 12B).

8. A composition, comprising:

(a) an inactive guide ribonucleic acid (RNA) comprising
(i) an unpaired hairpin loop domain,
(ii) a paired stem domain located adjacent to the unpaired hairpin loop domain and comprising a first subdomain complementary to and bound to a second subdomain,
(iii) an unpaired toehold domain contiguous with the first subdomain of the paired stem domain, and
(iv) an unpaired domain contiguous with the second subdomain of the paired stem domain that is capable of associating with an RNA-guided endonuclease when the first subdomain and second subdomain of (a)(ii) are not bound to each other; and optionally

(b) a trigger nucleic acid comprising (i) an unpaired subdomain complementary to the first subdomain of the paired stem domain of the inactive gRNA and (ii) an unpaired subdomain complementary to the toehold domain of the inactive gRNA (see, e.g., FIGS. 9A and 10A).

9. A composition, comprising:

(a) an inactive guide ribonucleic acid (RNA) comprising
(i) an unpaired hairpin loop domain,
(ii) a paired stem domain located adjacent to the unpaired hairpin loop domain and comprising a first subdomain complementary to and bound to a second subdomain, and
(iii) an unpaired domain contiguous with the second subdomain of the paired stem domain that is capable of associating with an RNA-guided endonuclease when the first subdomain and second subdomain of (a)(ii) are not bound to each other; and optionally

(b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA (see, e.g., FIGS. 9B and 10B).

10. A composition, comprising:

(a) a supporting ribonucleic acid (RNA) strand comprising, from 5′ to 3′, a first domain, a second domain, a third domain, a fourth domain, a fifth domain, a sixth domain, a seventh domain and an eight domain, wherein the second domain is complementary to the fourth domain to form a Csy4-specific hairpin, and the sixth domain is complementary to the eighth domain to form a Cas9-specific hairpin;

(b) a target RNA comprising, from 5′ to 3′, a first domain and a second domain, wherein the first domain of the target RNA is complementary to the second domain of the supporting RNA strand, and the second domain of the target RNA is complementary to the first domain of the supporting RNA strand; and

(c) a guide RNA strand comprising, from 5′ to 3′, a first domain containing a guide sequence, a second domain and a third domain, wherein the first domain of the guide RNA strand associates with Cas9 nuclease, the second domain of the guide RNA is complementary to the fifth domain of the supporting RNA strand, and the third domain of the guide RNA strand is complementary to the fourth domain of the supporting RNA strand (see, e.g., FIG. 14).

11. A composition, comprising:

(a) a supporting ribonucleic acid (RNA) strand comprising, from 5′ to 3′, 21 domains, wherein the 2^{nd} domain and the 3^{rd} domain are complementary to the 7^{th} domain and the 6^{th} domain, respectively, the 9^{th} domain is complementary to the 11^{th} domain, the 12^{th} domain and the 13^{th} domain are complementary to the 17^{th} domain and the 16^{th} domain, respectively, and the 19^{th} domain is complementary to the 21^{st} domain;

(b) a guide RNA strand comprising, from 5′ to 3′, a 1^{st} domain, a 2^{nd} domain and a 3^{rd} domain, wherein the 1^{st} domain of the guide RNA strand associates with a RNA-guided nuclease, the 2^{nd} domain of the guide RNA strand is complementary to the 18^{th} domain of the supporting RNA strand, and the third domain of the guide RNA strand is complementary to the 17^{th} domain of the supporting RNA strand; and

(c) an input RNA catalyst strand comprising, from 5′ to 3′, a 1^{st} domain, a 2^{nd} domain and a 3^{rd} domain, wherein the 1^{st} domain of the input RNA catalyst strand is complementary to the 3^{rd} domain of the supporting RNA strand, the 2^{nd} domain of the input RNA catalyst is complementary to the 2^{nd} domain of the supporting RNA strand, and the 3^{rd} domain of the input RNA catalyst is complementary to the 1^{st} domain of the supporting RNA strand (see, e.g., FIG. 15).

12. A composition, comprising:

(a) a first nucleic acid complex comprising
(i) a first nucleic acid strand comprising, from 5′ to 3′, a first domain, a second domain and a third domain, and
(ii) a second nucleic acid strand comprising, from 5′ to 3′, a first domain, a second domain and a third domain,

wherein the second domain of the second nucleic acid strand of (a)(ii) is complementary to the third domain of the first nucleic acid strand of (a)(i), and the third domain of the second nucleic acid strand of (a)(ii) is complementary to the second domain of the first nucleic acid strand of (a)(i);

(b) a second nucleic acid complex comprising
(i) a first nucleic acid strand comprising, from 5′ to 3′, a first domain and a second domain, each of which can associate with a DNA-guided nuclease, and
(ii) a second nucleic acid strand comprising, from 5′ to 3′, a first domain and a second domain,

wherein the first domain of the second nucleic acid strand of (b)(ii) is complementary second domain of the first nucleic acid strand of (a)(i), and wherein the second domain of the second nucleic acid strand of (b)(ii) is complementary to the second domain of the first nucleic acid strand of (b)(i) and is complementary to the first domain of the first nucleic acid strand of (a)(i); and

(c) a nucleic acid input strand comprising, from 5′ to 3′, a first domain, a second domain and a third domain,

wherein the first domain, second domain and third domain of the nucleic acid input strand are complementary to the third domain, second domain and first domain of the second nucleic acid strand of (a)(ii), respectively (see, e.g., FIG. 16).

13. A composition, comprising:

(a) a first nucleic acid complex comprising
(i) a first nucleic acid strand comprising, from 5′ to 3′, a first domain, a second domain, a third domain and a fourth domain, and
(ii) a second nucleic acid strand comprising, from 5′ to 3′, a first domain, a second domain and a third domain,

wherein the second domain of the second nucleic acid strand of (a)(ii) is complementary to the fourth domain of the first nucleic acid strand of (a)(i), and the third domain of the second nucleic acid strand of (a)(ii) is complementary to the third domain of the first nucleic acid strand of (a)(i);

(b) a second nucleic acid complex comprising
(i) a first nucleic acid strand comprising, from 5′ to 3′, a first domain and a second domain, each of which can associate with a DNA-guided nuclease, and
(ii) a second nucleic acid strand comprising, from 5′ to 3′, a first domain and a second domain, each of which can associate with a DNA-guided nuclease
(iii) a third nucleic acid strand comprising, from 5′ to 3′, a first domain, a second domain and a third domain,

wherein the first domain of the third nucleic acid strand of (b)(ii) is complementary to the third domain of the first nucleic acid strand of (a)(i), wherein the second domain of the third nucleic acid strand of (b)(iii) and is complementary to the second domain of the first nucleic acid strand of (a)(i), wherein the third domain of the third nucleic acid strand of (b)(iii) is complementary to the second domain of the first nucleic acid strand of (b)(i) and is complementary to the first domain of the first nucleic acid strand of (a)(i); and

(c) a nucleic acid input strand comprising, from 5′ to 3′, a first domain, a second domain and a third domain,

wherein the first domain, second domain and third domain of the nucleic acid input strand are complementary to the third domain, second domain and first domain of the second nucleic acid strand of (a)(ii), respectively (see, e.g., FIG. 17).

14. A composition, comprising

(a) a first nucleic acid complex comprising
(i) a first nucleic acid strand comprising, from 5′ to 3′, a first domain, a second domain and a third domain,
(ii) a second nucleic acid strand comprising, from 5′ to 3′, a first domain, a second domain and a third domain, and
(iii) a third nucleic acid strand comprising, from 5′ to 3′, a first domain, a second domain, a third domain and a fourth domain,

wherein the first domain and the second domain of the third nucleic acid strand of (a)(iii) are complementary to the first domain and the third domain of the second nucleic acid strand of (a)(ii), respectively, and wherein the third domain and the fourth domain of the third nucleic acid strand of (a)(iii) are complementary to the second and third domain of the first nucleic acid strand of (a)(i);

(b) a second nucleic acid complex comprising
(i) a first nucleic acid strand comprising a first domain, a second domain and a third domain, and
(ii) a second nucleic acid strand comprising a domain that is complementary to the second domain of the first nucleic acid strand of (b)(i) and can associate with a DNA-guided nuclease;

(c) a first nucleic acid input strand comprising, from 5′ to 3′, a first domain, a second domain and a third domain that are complementary to the third domain, second domain and first domain of the first nucleic acid strand of (a)(i), respectively; and

(d) a second nucleic acid input strand comprising, from 5′ to 3′, a first domain, a second domain and a third domain that are complementary to the third domain, second domain and first domain of the first nucleic acid strand of (a)(ii), respectively (see, e.g., FIG. 18).

15. A composition, comprising:

(a) a nucleic acid complex comprising
(i) a first nucleic acid strand comprising, from 5′ to 3′, a first domain and a second domain that can associate with a DNA-guided nuclease,
(ii) a second nucleic acid strand comprising, from 5′ to 3′, a first domain and a second domain, and
(iii) a third nucleic acid strand comprising, from 5′ to 3′, a first domain, a second domain, a third domain and a fourth domain,

wherein the first domain of the third nucleic acid strand of (a)(iii) is complementary to the first domain of the second nucleic acid strand of (a)(ii), and wherein the third domain of the third nucleic acid strand of (a)(iii) is complementary to the first domain of the first nucleic acid strand of (a)(i); and

(b) a first nucleic acid input strand comprising, from 5′ to 3′, a first domain and a second domain, or a second nucleic acid strand comprising, from 5′ to 3′, a first domain and a second domain,

wherein the first domain and the second domain of the first nucleic acid input strand of (b) are complementary to the fourth domain and the third domain of the third nucleic acid strand of (a)(iii), respectively, and wherein the first domain and the second domain of the second nucleic acid input strand of (b) are complementary to the second domain and the first domain of the third nucleic acid strand of (a)(iii), respectively (see, e.g., FIG. 19).

16. A method of using any one of the foregoing compositions to modify genomic nucleic acid in a cell.

17. The composition of any one of paragraphs 1-9, wherein the composition comprises both the inactive guide RNA and the trigger nucleic acid.

18. A cell comprising a nucleic acid encoding an inactive guide RNA described herein.

19. A cell comprising a nucleic acid encoding a trigger nucleic acid described herein.

20. The cell of paragraph 19, wherein the nucleic acid further encoding an inactive guide RNA described herein.

21. A vector comprising a nucleic acid encoding an inactive guide RNA described herein.

22. A vector comprising a nucleic acid encoding a trigger nucleic acid described herein.

23. The vector of paragraph 22, wherein the nucleic acid further encoding an inactive guide RNA described herein.

24. A nucleic acid molecule encoding an inactive guide RNA described herein.

25. A nucleic acid molecule encoding a trigger nucleic acid described herein.

26. The nucleic acid molecule of paragraph 25, wherein the nucleic acid molecule encodes an inactive guide RNA and the trigger nucleic acid.

27. A kit or composition comprising:
(a) an inactive guide RNA as described herein;
(b) a trigger nucleic acid as described herein; and
(c) an RNA-guided nuclease.

28. A kit or composition comprising:
(a) in active guide RNA as described herein; and
(b) a trigger nucleic acid as described herein.

### EXAMPLES

### Example 1

In vitro cleavage assays were performed to test exemplary switch-gRNA designs, as follows:

1. RNA molecules were transcribed in vitro from synthetic DNA.

2. Streptococcus pyogenes Cas9 protein was obtained (PNA bio).

3. In vitro cleavage reactions were performed, as follows:
mix 150 ng Cas9 protein, 75 ng Target DNA (cf. below), 100 ng of RNA (sgRNA, switch-gRNA, trigger) in 100 mM NaCl, 50 mM Tris-HCl, 10 mM MgCl₂, 100 μg/ml BSA (pH 7.9@25° C.); and
incubate for 1 hour at 37° C., then 10 min at 65° C. to inactivate Cas9.

4. Reaction products were analyzed by denaturing gel electrophoresis (TBE-Urea PAGE gel).

Target DNA sequence (synthetic double-stranded DNA comprising protospacers 1 and 2) used in cleavage assays:

(SEQ ID NO: 38)
TCGAGGTCGCAGGTATCTTTAGGGTAGACATCTTACGGGGAGAGCACTA
ACCCATCAACCTGTACGGTTCACAGTCGGTCACATTGGGGTACTCCTTG
GCTTTTCCGCTTGGCCCGGTCTGTTAGGCCCCCGTAGCGTGAGTTTCGG
CCCTGTGCTGCCCAGTACGAGCTGATTGAACATGGGTTGGGGGCCTCGA
ACCGTCGAGGACCCCATAGTACCTCGGAGACCAAGTGATTTCTTCTTGC
GCTTTTTTGGGAACTATTTCGGGGGGCGAGCCCTCATCGTCTCTTCGCG
GATGACTCAACACGCTAGGGACGTGACCGCGGGCAGTCGATTCCTTCGA
TGGTTATAAATCGCGTGCGCTCTATTTGACGATCCCTTGGCGCAGAGGT
GCTGGCCACGTGCTAAATTAAAGCGGCTGCACTACTGTAAGGTCCGTCA
CGGATGTGATTCCACTCTATTGGACGGCGGCCAAAGACTAAAGACCATC
TCGCTCGAGA

Guide RNA sequences were programmed to target:
Protospacer 1 (in bold, GATTTCTTCTTGCGCTTTTT) (SEQ ID NO: 39)
Protospacer 2 (in bold, GGTTCACAGTCGGTCACATT) (SEQ ID NO: 9)
Protospacer adjacent motif (PAM) are underlined.

### Example 2

Interfacing the transcriptome with programmable CRISPR-Cas logic functions enables multiplex sensing of endogenous RNA sequences in living cells, and actuates a wide range of outputs based on user-specified CRISPR programs. Examples include DNA encoding memories, recording and tracking a series of events at the single-cell level, activating nucleic acid synthesis from endogenous or synthetic genes, dynamic cellular reprogramming, and generating detectable signals. A multiplex RNA-sensing CRISPR-Cas system enables continuous monitoring of multiple input/output (I/O) in bacteria and eukaryotes.

The present disclosure provide a method for sgRNA engineering based on the concept of toehold-mediated strand displacement. Consider the hairpin structure in FIG. 5A, where sequence 1 is protected by its complementary sequence 1*. Here, sequence x serves as a toehold so that if the trigger sequence is present, it can bind the toehold and unfold the hairpin by branch migration, resulting in a structure that exposes sequence y. A strand displacement mechanism has been assimilated to a molecular switch, as described herein, whereby a relevant sequence (e.g., sequence y) is masked and exposed in the presence of the cognate nucleic acid trigger.

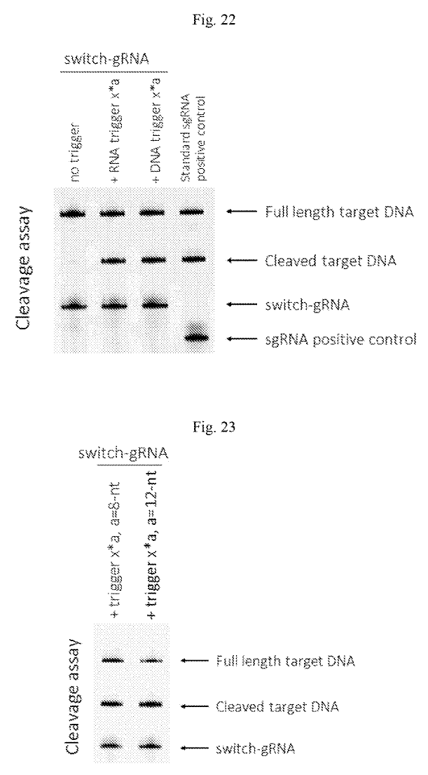

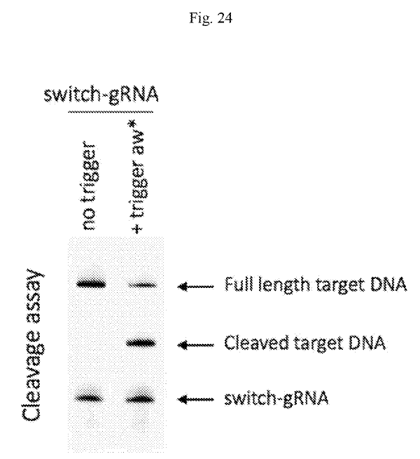

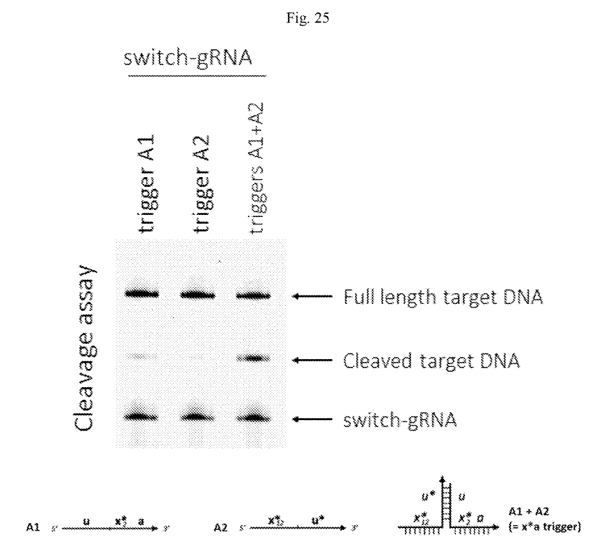

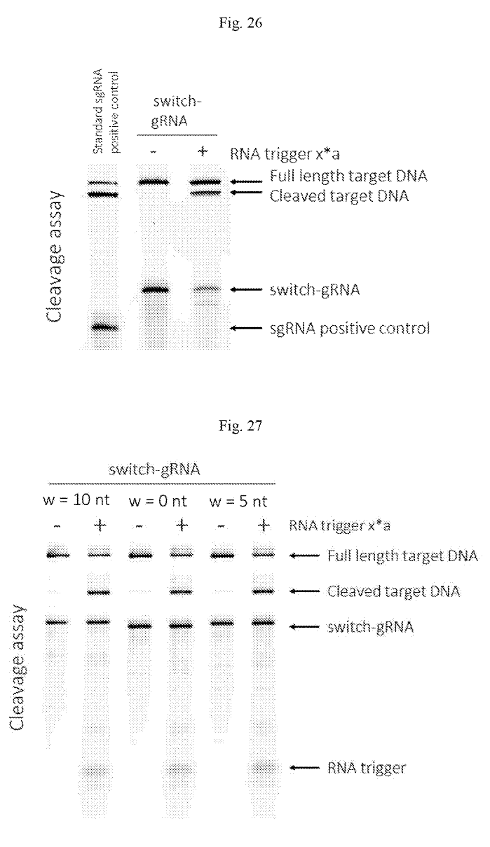

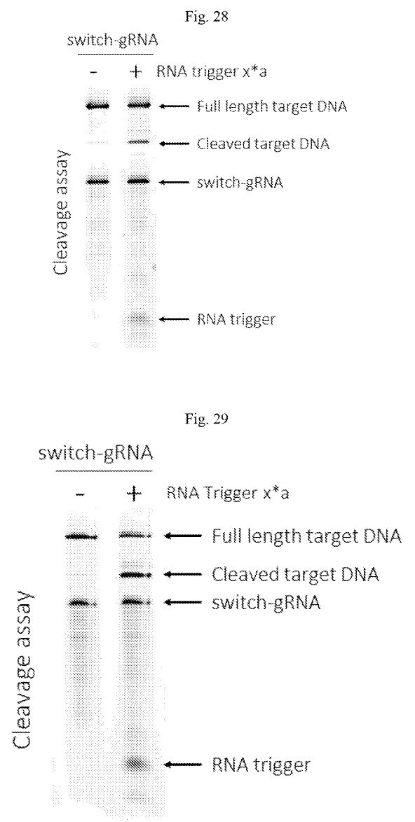

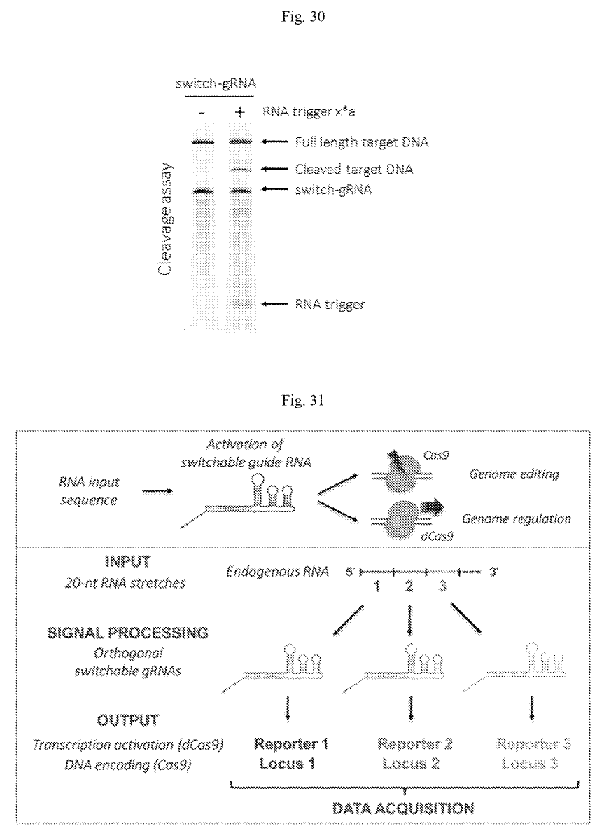

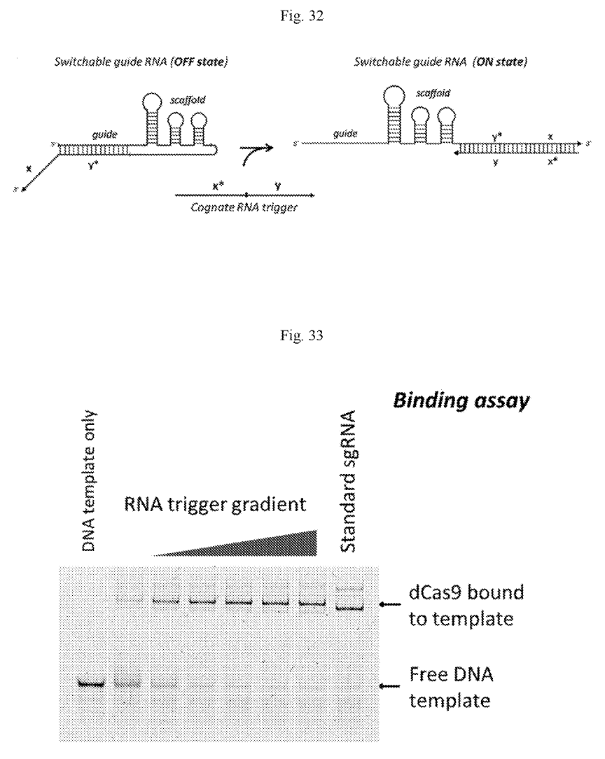

Since many biological systems require binding to specific RNA elements, this strand displacement mechanism offers a unique solution to modulate these systems in vivo. We engineered a switchable guide RNA by implementing a toehold-mediated strand displacement mechanism into the single-guide RNA of CRISPR-Cas9. The switchable guide RNA was designed by masking the guide sequence of the sgRNA with the protector strand y* to inactivate CRISPR activity in the absence of the cognate RNA trigger (FIG. 31). Given the correct RNA trigger sequence, the strand displacement mechanism exposes the guide sequence thereby activating the guide RNA and the desired CRISPR activity. Switchable guide RNA were successfully tested in vitro using cleavage and binding assays, demonstrating that CRISPR cleavage and binding activities are both controlled by the RNA trigger (e.g., FIGS. 22 and 32).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
(a) an inactive guide ribonucleic acid (RNA) comprising a 5′ end, a 3′ end, and
(i) an unpaired hairpin loop domain,
(ii) a paired stem domain located adjacent to the unpaired hairpin loop domain and comprising a first subdomain complementary to and bound to a second subdomain,
(iii) an unpaired toehold domain contiguous with the first subdomain of the paired stem domain, and
(iv) an unpaired guide domain contiguous with the second subdomain of the paired stem domain that is capable of associating with an RNA-guided endonuclease when the first subdomain and second subdomain of (a)(ii) are not bound to each other; and
(b) a trigger nucleic acid comprising (i) an unpaired subdomain complementary to the first subdomain of the paired stem domain of the inactive gRNA and (ii) an unpaired subdomain complementary to the toehold domain of the inactive gRNA.

2. The composition of claim 1 further comprising the RNA-guided endonuclease.

3. The composition of claim 1 or 2 further comprising a target nucleic acid.

4. The composition of claim 3, wherein the guide domain of (a)(iv) comprises a nucleotide sequence that is complementary to the target nucleic acid.

5. The composition of any one of claims 1-4, wherein the RNA-guided endonuclease is Cas9, Cpf1 or C2c2.

6. The composition of any one of claims 1-5, wherein the unpaired toehold domain is located at the 5′ end of the inactive guide RNA.

7. The composition of claim 6, wherein the 3′ end of the inactive guide RNA comprises a scaffold formed by intramolecular nucleotide base pairing.

8. The composition of claim 6 or 7, wherein the unpaired subdomain (i) of the trigger nucleic acid is upstream from the unpaired subdomain (ii) of the trigger nucleic acid.

9. The composition of any one of claims 1-5, wherein the unpaired toehold domain is located at the 3′ end of the inactive guide RNA.

10. The composition of claim 8, wherein the unpaired hairpin loop domain of the inactive guide RNA comprises a scaffold formed by intramolecular nucleotide base pairing.

11. The composition of claim 9 or 10, wherein the unpaired subdomain (ii) of the trigger nucleic acid is upstream from the unpaired subdomain (i) of the trigger nucleic acid.

12. A method comprising incubating in reaction buffer in the presence of a target nucleic acid and an RNA-guided nuclease the composition of any one of claims 1-11 to produce an active gRNA that associates with the RNA-guided nuclease and binds to the target nucleic acid.

13. A composition, comprising:
(a) an inactive guide ribonucleic acid (RNA) comprising a 5′ end, a 3′ end, and
(i) an unpaired hairpin loop domain,
(ii) a paired stem domain located adjacent to the unpaired hairpin loop domain and comprising a first subdomain complementary to and bound to a second subdomain, and
(iii) an unpaired guide domain contiguous with the second subdomain of the paired stem domain that is capable of associating with an RNA-guided endonuclease when the first subdomain and second subdomain of (a)(ii) are not bound to each other; and
(b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA.

14. The composition of claim 13 further comprising the RNA-guided endonuclease.

15. The composition of claim 13 or 14 further comprising a target nucleic acid.

16. The composition of claim 15, wherein the guide domain of (a)(iii) comprises a nucleotide sequence that is complementary to the target nucleic acid.

17. The composition of any one of claims 13-16, wherein the RNA-guided endonuclease is Cas9, Cpf1 or C2c2.

18. The composition of any one of claims 13-17, wherein the first domain is located at the 5′ end of the inactive guide RNA.

19. The composition of claim 18, wherein the 3′ end of the inactive guide RNA comprises a scaffold formed by intramolecular nucleotide base pairing.

20. The composition of claim 18 or 19, wherein the unpaired subdomain (i) of the trigger nucleic acid is upstream from the unpaired subdomain (ii) of the trigger nucleic acid.

21. The composition of any one of claims 13-17, wherein the first domain is located at the 3′ end of the inactive guide RNA.

22. The composition of claim 21, wherein the unpaired hairpin loop domain of the inactive guide RNA comprises a scaffold formed by intramolecular nucleotide base pairing.

23. The composition of claim 21 or 22, wherein the unpaired subdomain (ii) of the trigger nucleic acid is upstream from the unpaired subdomain (i) of the trigger nucleic acid.

24. A method comprising incubating in reaction buffer in the presence of a target nucleic acid and an RNA-guided nuclease the composition of any one of claims 13-23 to produce an active gRNA that associates with the RNA-guided nuclease and binds to the target nucleic acid.

25. A composition, comprising:
(a) an inactive guide ribonucleic acid (RNA) comprising a 5′ end, a 3′ end, and
(i) an unpaired hairpin loop domain,
(ii) a paired stem domain located adjacent to the unpaired hairpin loop domain and comprising a first subdomain complementary to and bound to a second guide subdomain that, when not bound to the first subdomain, associates with an RNA-guided endonuclease, and
(iii) an unpaired toehold domain contiguous with the first subdomain of the paired stem domain; and
(b) a trigger nucleic acid comprising (i) an unpaired subdomain complementary to the first subdomain of the paired stem domain of the inactive gRNA and (ii) an unpaired subdomain complementary to the toehold domain of the inactive gRNA.

26. The composition of claim 25 further comprising the RNA-guided endonuclease.

27. The composition of claim 25 or 26 further comprising a target nucleic acid.

28. The composition of claim 27, wherein the second subdomain of (a)(ii) comprises a nucleotide sequence that is complementary to a target gene of interest.

29. The composition of any one of claims 25-28, wherein the RNA-guided endonuclease is Cas9, Cpf1 or C2c2.

30. The composition of any one of claims 25-29, wherein the unpaired toehold domain is located at the 5′ end of the inactive guide RNA.

31. The composition of claim 30, wherein the 3′ end of the inactive guide RNA comprises a scaffold formed by intramolecular nucleotide base pairing.

32. The composition of claim 30 or 31, wherein the unpaired subdomain (i) of the trigger nucleic acid is upstream from the unpaired subdomain (ii) of the trigger nucleic acid.

33. The composition of any one of claims 25-29, wherein the unpaired toehold domain is located at the 3′ end of the inactive guide RNA.

34. The composition of claim 33, wherein the unpaired hairpin loop domain of the inactive guide RNA comprises a scaffold formed by intramolecular nucleotide base pairing.

35. The composition of claim 33 or 34, wherein the unpaired subdomain (ii) of the trigger nucleic acid is upstream from the unpaired subdomain (i) of the trigger nucleic acid.

36. A method comprising incubating in reaction buffer in the presence of a target nucleic acid and an RNA-guided nuclease the composition of any one of claims 25-35 to produce an active gRNA that associates with the RNA-guided nuclease and binds to the target nucleic acid.

37. A composition, comprising:
(a) an inactive guide ribonucleic acid (RNA) comprising a 5′ end, a 3′ end, and
(i) an unpaired hairpin loop domain,
(ii) a paired stem domain located adjacent to the unpaired hairpin loop domain and comprising a first subdomain complementary to and bound to a second guide subdomain that, when not bound to the first subdomain, associates with an RNA-guided endonuclease, and
(iii) an unpaired toehold domain contiguous with the first subdomain of the paired stem domain; and
(b) a first trigger nucleic acid comprising
(i) an unpaired subdomain complementary to the first subdomain of the paired stem domain of the inactive gRNA and
(ii) an unpaired subdomain; and
(c) a second trigger nucleic acid comprising
(i) an unpaired subdomain complementary to the unpaired subdomain of (b)(ii) and
(ii) an unpaired subdomain complementary to the toehold domain of the inactive gRNA.

38. The composition of claim 37 further comprising the RNA-guided endonuclease.

39. The composition of claim 37 or 38 further comprising a target nucleic acid.

40. The composition of claim 39, wherein the second subdomain of (a)(ii) comprises a nucleotide sequence that is complementary to the target nucleic acid.

41. The composition of any one of claims 37-40, wherein the RNA-guided endonuclease is Cas9, Cpf1 or C2c2.

42. A composition, comprising:
(a) an inactive guide ribonucleic acid (RNA) comprising a 5′ end, a 3′ end, and
(i) an unpaired hairpin loop domain, and
(ii) a paired stem domain located adjacent to the unpaired hairpin loop domain and comprising a first subdomain complementary to and bound to a second guide subdomain that, when not bound to the first subdomain, associates with an RNA-guided endonuclease; and
(b) a trigger nucleic acid comprising (i) an unpaired subdomain complementary to the hairpin loop domain of the inactive gRNA and (ii) an unpaired subdomain complementary to the first subdomain of the paired stem domain of the inactive gRNA.

43. The composition of claim 42 further comprising the RNA-guided endonuclease.

44. The composition of claim 42 or 43 further comprising a target nucleic acid.

45. The composition of claim 44, wherein the second subdomain of (a)(ii) comprises a nucleotide sequence that is complementary to the target nucleic acid.

46. The composition of any one of claims 42-45, wherein the RNA-guided endonuclease is Cas9, Cpf1 or C2c2.

47. The composition of any one of claims 42-46, wherein the first domain is located at the 5′ end of the inactive guide RNA.

48. The composition of claim 47, wherein the 3′ end of the inactive guide RNA comprises a scaffold formed by intramolecular nucleotide base pairing.

49. The composition of claim 47 or 48, wherein the unpaired subdomain (i) of the trigger nucleic acid is upstream from the unpaired subdomain (ii) of the trigger nucleic acid.

50. The composition of any one of claims 42-46, wherein the first domain is located at the 3′ end of the inactive guide RNA.

51. The composition of claim 49, wherein the unpaired hairpin loop domain of the inactive guide RNA comprises a scaffold formed by intramolecular nucleotide base pairing.

52. The composition of claim 50 or 51, wherein the unpaired subdomain (ii) of the trigger nucleic acid is upstream from the unpaired subdomain (i) of the trigger nucleic acid.

53. A method comprising incubating in reaction buffer in the presence of a target nucleic acid and an RNA-guided nuclease the composition of any one of claims 42-52 to produce an active gRNA that associates with the RNA-guided nuclease and binds to the target nucleic acid.

54. A composition, comprising:
(a) an inactive guide ribonucleic acid (RNA) comprising a 5′ end, a 3′ end, and
(i) an unpaired hairpin loop domain,
(ii) a paired stem domain located adjacent to the unpaired hairpin loop domain and comprising a first subdomain contiguous with a second subdomain, and a third subdomain contiguous with a fourth guide subdomain, wherein the first subdomain and the second subdomain are respectively complementary to and bound to the third subdomain and the fourth guide subdomain, and wherein fourth guide subdomain, when not bound to the second subdomain, associates with an RNA-guided endonuclease, and
(iii) an unpaired toehold domain contiguous with the first subdomain of the paired stem domain; and
(b) a trigger nucleic acid comprising (i) an unpaired subdomain complementary to the first subdomain of the paired stem domain of the inactive gRNA, (ii) an unpaired subdomain complementary to the second subdomain of the paired stem domain of the inactive gRNA, and (iii) an unpaired subdomain complementary to the toehold domain of the inactive gRNA.

55. The composition of claim 54 further comprising the RNA-guided endonuclease.

56. The composition of claim 54 or 55 further comprising a target nucleic acid.

57. The composition of any one of claims 54-56, wherein the fourth guide subdomain of (a)(ii) comprises a nucleotide sequence that is complementary to a target gene of interest.

58. The composition of any one of claims 54-57, wherein the RNA-guided endonuclease is Cas9, Cpf1 or C2c2.

59. The composition of any one of claims 54-58, wherein the unpaired toehold domain is located at the 3′ end of the inactive guide RNA.

60. The composition of claim 59, wherein the unpaired hairpin loop domain of the inactive guide RNA comprises a scaffold formed by intramolecular nucleotide base pairing.

61. The composition of claim 59 or 60, wherein the unpaired subdomain (i) of the trigger nucleic acid is downstream from the unpaired subdomain (iii) of the trigger nucleic acid and upstream from the unpaired subdomain (ii) of the trigger nucleic acid.

62. The composition of any one of claims 54-58, wherein the unpaired toehold domain is located at the 5′ end of the inactive guide RNA.

63. The composition of claim 59, wherein the 3′ end of the inactive guide RNA comprises a scaffold formed by intramolecular nucleotide base pairing.

64. The composition of claim 62 or 63, wherein the unpaired subdomain (ii) of the trigger nucleic acid is downstream from the unpaired subdomain (i) of the trigger nucleic acid and upstream from the unpaired subdomain (iii) of the trigger nucleic acid.

65. A method comprising incubating in reaction buffer in the presence of a target nucleic acid and an RNA-guided nuclease the composition of any one of claims 54-64 to produce an active gRNA that associates with the RNA-guided nuclease and binds to the target nucleic acid.

66. A composition, comprising:
(a) an inactive guide ribonucleic acid (RNA) comprising a 5′ end, a 3′ end, and
(i) an unpaired hairpin loop domain, and
(ii) a paired stem domain located adjacent to the unpaired hairpin loop domain and comprising a first subdomain contiguous with a second subdomain, and a third subdomain contiguous with a fourth subdomain, wherein the first subdomain and the second subdomain are respectively complementary to and bound to the third subdomain and the fourth subdomain, and wherein fourth subdomain, when not bound to the second subdomain, associates with an RNA-guided endonuclease; and
(b) a trigger nucleic acid comprising (i) an unpaired subdomain complementary to the hairpin loop domain of the inactive gRNA, (ii) an unpaired subdomain complementary to the first subdomain of the paired stem domain of the inactive gRNA, and (iii) an unpaired subdomain complementary to the second subdomain of the paired stem domain of the inactive gRNA.

67. The composition of claim 66 further comprising the RNA-guided endonuclease.

68. The composition of claim 66 or 67 further comprising a target nucleic acid.

69. The composition of any one of claims 66-68, wherein the fourth guide subdomain of (a)(ii) comprises a nucleotide sequence that is complementary to a target gene of interest.

70. The composition of any one of claims 66-69, wherein the RNA-guided endonuclease is Cas9, Cpf1 or C2c2.

71. The composition of any one of claims 66-70, wherein the first domain is located at the 3′ end of the inactive guide RNA.

72. The composition of claim 71, wherein the unpaired hairpin loop domain of the inactive guide RNA comprises a scaffold formed by intramolecular nucleotide base pairing.

73. The composition of claim 71 or 72, wherein the unpaired subdomain (ii) of the trigger nucleic acid is downstream from the unpaired subdomain (i) of the trigger nucleic acid and upstream from the unpaired subdomain (iii) of the trigger nucleic acid.

74. The composition of any one of claims 66-70, wherein the second domain is located at the 5′ end of the inactive guide RNA.

75. The composition of claim 74, wherein the 3′ end of the inactive guide RNA comprises a scaffold formed by intramolecular nucleotide base pairing.

76. The composition of claim 74 or 75, wherein the unpaired subdomain (i) of the trigger nucleic acid is downstream from the unpaired subdomain (iii) of the trigger nucleic acid and upstream from the unpaired subdomain (ii) of the trigger nucleic acid.

77. A method comprising incubating in reaction buffer in the presence of a target nucleic acid and an RNA-guided nuclease the composition of any one of claims 66-76 to produce an active gRNA that associates with the RNA-guided nuclease and binds to the target nucleic acid.

78. A composition, comprising:
(a) a supporting ribonucleic acid (RNA) strand comprising, from 5′ to 3′, a first domain, a second domain, a third domain, a fourth domain, a fifth domain, a sixth domain, a seventh domain and an eight domain, wherein the second domain is complementary to the fourth domain to form a Csy4-specific hairpin, and the sixth domain is complementary to the eighth domain to form a Cas9-specific hairpin;
(b) a target RNA comprising, from 5′ to 3′, a first domain and a second domain, wherein the first domain of the target RNA is complementary to the second domain of the supporting RNA strand, and the second domain of the target RNA is complementary to the first domain of the supporting RNA strand; and
(c) a guide RNA strand comprising, from 5′ to 3′, a first domain containing a guide sequence, a second domain and a third domain, wherein the first domain of the guide RNA strand associates with Cas9 nuclease, the second domain of the guide RNA is complementary to the fifth domain of the supporting RNA strand, and the third domain of the guide RNA strand is complementary to the fourth domain of the supporting RNA strand.

79. The composition of claim 78 further comprising Csy4 nuclease.

80. The composition of claim 78 or 79 further comprising Cas9 nuclease.

81. The composition of any one of claims 78-80 further comprising a target nucleic acid.

82. A composition, comprising:
(a) a supporting ribonucleic acid (RNA) strand comprising, from 5′ to 3′, 21 domains, wherein the 2^{nd} domain and the 3^{rd} domain are complementary to the 7^{th} domain and the 6^{th} domain, respectively, the 9^{th} domain is complementary to the 11^{th} domain, the 12^{th} domain and the 13^{th} domain are complementary to the 17^{th} domain and the 16^{th} domain, respectively, and the 19^{th} domain is complementary to the 21^{st} domain;
(b) a guide RNA strand comprising, from 5′ to 3′, a 1^{st} domain, a 2^{nd} domain and a 3^{rd} domain, wherein the 1^{st} domain of the guide RNA strand associates with a RNA-guided nuclease, the 2^{nd} domain of the guide RNA strand is complementary to the 18^{th} domain of the supporting RNA strand, and the third domain of the guide RNA strand is complementary to the 17^{th} domain of the supporting RNA strand; and
(c) an input RNA catalyst strand comprising, from 5′ to 3′, a 1^{st} domain, a 2^{nd} domain and a 3^{rd} domain, wherein the 1^{st} domain of the input RNA catalyst strand is complementary to the 3^{rd} domain of the supporting RNA strand, the 2^{nd} domain of the input RNA catalyst is complementary to the 2^{nd} domain of the supporting RNA strand, and the 3rd domain of the input RNA catalyst is complementary to the 1^{St} domain of the supporting RNA strand.

83. The composition of claim 82 further comprising the RNA-guided nuclease.

84. The composition of claim 82 or 83 further comprising a target nucleic acid.

85. A composition, comprising:
(a) a first nucleic acid complex comprising
(i) a first nucleic acid strand comprising, from 5′ to 3′, a first domain, a second domain and a third domain, and
(ii) a second nucleic acid strand comprising, from 5′ to 3′, a first domain, a second domain and a third domain,
wherein the second domain of the second nucleic acid strand of (a)(ii) is complementary to the third domain of the first nucleic acid strand of (a)(i), and the third domain of the second nucleic acid strand of (a)(ii) is complementary to the second domain of the first nucleic acid strand of (a)(i);
(b) a second nucleic acid complex comprising
(i) a first nucleic acid strand comprising, from 5′ to 3′, a first domain and a second domain, each of which can associate with a DNA-guided nuclease, and
(ii) a second nucleic acid strand comprising, from 5′ to 3′, a first domain and a second domain,
wherein the first domain of the second nucleic acid strand of (b)(ii) is complementary second domain of the first nucleic acid strand of (a)(i), and wherein the second domain of the second nucleic acid strand of (b)(ii) is complementary to the second domain of the first nucleic acid strand of (b)(i) and is complementary to the first domain of the first nucleic acid strand of (a)(i); and
(c) a nucleic acid input strand comprising, from 5′ to 3′, a first domain, a second domain and a third domain,
wherein the first domain, second domain and third domain of the nucleic acid input strand are complementary to the third domain, second domain and first domain of the second nucleic acid strand of (a)(ii), respectively.

86. The composition of claim 85 further comprising a DNA-guided nuclease.

87. The composition of claim 85 or 86 further comprising a target nucleic acid.

88. A composition, comprising:
(a) a first nucleic acid complex comprising
(i) a first nucleic acid strand comprising, from 5′ to 3′, a first domain, a second domain, a third domain and a fourth domain, and
(ii) a second nucleic acid strand comprising, from 5′ to 3′, a first domain, a second domain and a third domain,
wherein the second domain of the second nucleic acid strand of (a)(ii) is complementary to the fourth domain of the first nucleic acid strand of (a)(i), and the third domain of the second nucleic acid strand of (a)(ii) is complementary to the third domain of the first nucleic acid strand of (a)(i);
(b) a second nucleic acid complex comprising
(i) a first nucleic acid strand comprising, from 5′ to 3′, a first domain and a second domain, each of which can associate with a DNA-guided nuclease, and
(ii) a second nucleic acid strand comprising, from 5′ to 3′, a first domain and a second domain, each of which can associate with a DNA-guided nuclease
(iii) a third nucleic acid strand comprising, from 5′ to 3′, a first domain, a second domain and a third domain,
wherein the first domain of the third nucleic acid strand of (b)(ii) is complementary to the third domain of the first nucleic acid strand of (a)(i), wherein the second domain of the third nucleic acid strand of (b)(iii) and is complementary to the second domain of the first nucleic acid strand of (a)(i), wherein the third domain of the third nucleic acid strand of (b)(iii) is complementary to the second domain of the first nucleic acid strand of (b)(i) and is complementary to the first domain of the first nucleic acid strand of (a)(i); and
(c) a nucleic acid input strand comprising, from 5′ to 3′, a first domain, a second domain and a third domain,
wherein the first domain, second domain and third domain of the nucleic acid input strand are complementary to the third domain, second domain and first domain of the second nucleic acid strand of (a)(ii), respectively.

89. The composition of claim 88 further comprising a DNA-guided nuclease.

90. The composition of claim 88 or 89 further comprising a target nucleic acid.

91. A composition, comprising
(a) a first nucleic acid complex comprising
(i) a first nucleic acid strand comprising, from 5′ to 3′, a first domain, a second domain and a third domain,
(ii) a second nucleic acid strand comprising, from 5′ to 3′, a first domain, a second domain and a third domain, and
(iii) a third nucleic acid strand comprising, from 5′ to 3′, a first domain, a second domain, a third domain and a fourth domain,
wherein the first domain and the second domain of the third nucleic acid strand of (a)(iii) are complementary to the first domain and the third domain of the second nucleic acid strand of (a)(ii), respectively, and wherein the third domain and the fourth domain of the third nucleic acid strand of (a)(iii) are complementary to the second and third domain of the first nucleic acid strand of (a)(i);
(b) a second nucleic acid complex comprising
(i) a first nucleic acid strand comprising a first domain, a second domain and a third domain, and
(ii) a second nucleic acid strand comprising a domain that is complementary to the second domain of the first nucleic acid strand of (b)(i) and can associate with a DNA-guided nuclease;
(c) a first nucleic acid input strand comprising, from 5′ to 3′, a first domain, a second domain and a third domain that are complementary to the third domain, second domain and first domain of the first nucleic acid strand of (a)(i), respectively; and
(d) a second nucleic acid input strand comprising, from 5′ to 3′, a first domain, a second domain and a third domain that are complementary to the third domain, second domain and first domain of the first nucleic acid strand of (a)(ii), respectively.

92. The composition of claim 91 further comprising a DNA-guided nuclease.

93. The composition of claim 91 or 92 further comprising a target nucleic acid.

94. A composition, comprising:
(a) a nucleic acid complex comprising
(i) a first nucleic acid strand comprising, from 5′ to 3′, a first domain and a second domain that can associate with a DNA-guided nuclease,
(ii) a second nucleic acid strand comprising, from 5′ to 3′, a first domain and a second domain, and
(iii) a third nucleic acid strand comprising, from 5′ to 3′, a first domain, a second domain, a third domain and a fourth domain,
wherein the first domain of the third nucleic acid strand of (a)(iii) is complementary to the first domain of the second nucleic acid strand of (a)(ii), and wherein the third domain of the third nucleic acid strand of (a)(iii) is complementary to the first domain of the first nucleic acid strand of (a)(i); and
(b) a first nucleic acid input strand comprising, from 5′ to 3′, a first domain and a second domain, or a second nucleic acid strand comprising, from 5′ to 3′, a first domain and a second domain,
wherein the first domain and the second domain of the first nucleic acid input strand of (b) are complementary to the fourth domain and the third domain of the third nucleic acid strand of (a)(iii), respectively, and wherein the first domain and the second domain of the second nucleic acid input strand of (b) are complementary to the second domain and the first domain of the third nucleic acid strand of (a)(iii), respectively.

95. The composition of claim 94 further comprising a DNA-guided nuclease.

96. The composition of claim 94 or 95 further comprising a target nucleic acid.

97. A method of using any one of the foregoing compositions to modify genomic nucleic acid in a cell.

98. A cell comprising any one of the foregoing compositions.

99. The cell of claim 98, wherein the cell is a prokaryotic cell or a eukaryotic cell.
